(19) United States
(12) Patent Application Publication
(10) Pub. No.: US 2026/0045317 A1
(43) Pub. Date: Feb. 12, 2026

(54) METHOD FOR SIMULATING ELECTROPHYSIOLOGICAL RESPONSES

(71) Applicant: Morphoceuticals, Inc., Medford, MA (US)

(72) Inventors: Cathal McLoughlin, Medford, MA (US); Hananel Hazan, Medford, MA (US); Nicolas Palacios-Prado, Medford, MA (US)

(73) Assignee: Morphoceuticals, Inc., Medford, MA (US)

(21) Appl. No.: 19/295,115

(22) Filed: Aug. 8, 2025

Related U.S. Application Data
(60) Provisional application No. 63/681,551, filed on Aug. 9, 2024.

(51) Int. Cl. G16B 5/00

(52) U.S. Cl. CPC G16B 5/00

(57) ABSTRACT

A differentiable bioelectric tissue simulator is provided. Methods of using a bioelectric tissue simulator are also provided.

### STATEMENT OF PRIORITY

This application claims priority to U.S. provisional application No. 63/681,551, filed Aug. 9, 2024, the contents of which are herein incorporated by reference in its entirety.

### TECHNICAL FIELD

The present disclosure relates generally to software for simulating electrophysiological responses of cells and tissues.

### BACKGROUND

The following description of the background of the present technology is provided simply as an aid in understanding the present technology and is not admitted to describe or constitute prior art to the present technology.

Bioelectric cell properties have been revealed as powerful targets for modulating stem cell function, regenerative response, developmental patterning, and tumor reprograming. Spatio-temporal distributions of endogenous resting potential, ion flows, and electric fields are influenced not only by the genome and external signals but also by their own intrinsic dynamics. Ion channels and electrical synapses (gap junctions) both determine and are, themselves, gated by cellular resting potential. Thus, the origin and progression of bioelectric patterns in multicellular tissues is complex, which hampers the rational control of voltage distributions for biomedical interventions.

On the scale of single cells, the Vmem spanning every living cell's plasma membrane is a demonstrated regulator of key processes, such as cell proliferation (Blackiston et al., 2009), programed cell death (Boutillier et al., 1999; Wang et al., 1999), and differentiation (Ng et al., 2010), and is known to be a factor in the activation of immune cells (Bronstein-Sitton, 2004). For example, despite the action of growth factors, stem cells have been inhibited from differentiation by preventing the cells from developing a hyperpolarized Vmem (Sundelacruz et al., 2008). The bioelectric properties of single cells are fairly well-understood (Lodish et al., 2000; Wright, 2004). However, bioelectric states often regulate large-scale anatomical properties, such as axial polarity (Marsh and Beams, 1952; Beane et al., 2011), organ size (Perathoner et al., 2014) and shape (Beane et al., 2013), and induction of formation of whole appendages (Adams et al., 2007; Tseng et al., 2010). Moreover, pattern control involves long-range coordination of bioelectric states. In metastatic conversion (Morokuma et al., 2008; Blackiston et al., 2011; Lobikin et al., 2012), tumor suppression (Chernet and Levin, 2014; Chernet et al., 2015), brain size regulation (Pai et al., 2015), and head-tail polarity in planarian regeneration (Beane et al., 2011), the patterning outcome in one region of the animal is a function of the bioelectric states of both local and remote cells. Thus, it is imperative to understand not only how ion channel and pump activity controls single-cell electrical properties but also how electrical gradients self-organize, propagate, and evolve in multicellular networks. Moreover, understanding the origin of developmental order also requires that we understand how tissue-level gradients of bioelectric properties arise.

In a multicellular collective, endogenous patterns of Vmem and electric fields provide positional information and achieve long-range coordination of cell activity. As in the central nervous system, this occurs because cells in a tissue are not isolated, but are electrochemically connected (and, therefore, communicating) in several ways, including intracellular channels known as gap junctions [GJ (Goodenough and Paul, 2009)], and by ephaptic coupling created by local field potentials, which enable one cell's Vmem activity to influence that of its neighbor's (Zhou et al., 2012). These connections between cells create bioelectrical circuits involving long-range signal patterns through whole structures, which have been determined crucial for developing embryos (Jaffe, 1981; Hotary and Robinson, 1990; Hotary and Robertson, 1994; Shi and Borgens, 1995), normal limb development of animals (Altizer et al., 2001), healing of wounds (Nuccitelli, 1992, 2003a; McCaig et al., 2005; Zhao, 2009), and even in continuous tumor suppression in adult animals (Chernet and Levin, 2013, 2014). The ability for cells to couple and communicate makes local changes to cell Vmem relevant in terms of long-range signals capable of affecting the whole. Likewise, the inability for cells to form communication networks, for instance, due to improper expression or function of GJ connections, is observed in disease processes, such as cancer (Leithe et al., 2006; Trosko, 2007). Even briefly altering the bioelectric connectivity of a cellular network enables rewriting of an organism's target morphology. For example, genomically normal fragments of planarian flatworms can be induced to regenerate heads with shapes and internal anatomy belonging to other extant species (Emmons-Bell et al., 2015), or changed to a two-headed form that regenerates with two heads in perpetuity, illustrating the ability to stably re-wire bioelectric circuits with permanent changes to the overall anatomy (Oviedo et al., 2010).

Another important bioelectrical signal relevant to multicellular clusters is a voltage gradient known as the trans-epithelial potential (TEP), which forms at the outer boundary of an organ or organism. The TEP is also implicated in normal developmental processes (Shi and Borgens, 1995), wound healing (Zhao, 2009), and disease processes, such as cystic fibrosis (Hay and Geddes, 1985), fungal infection (Gow and Morris, 1995), inflammation, and cancer (Soler et al., 1999). The TEP is created when multicellular structures develop impermeable tight junctions (TJ) between cells at the exterior boundary (Hay and Geddes, 1985); disruptions to this process induce electric fields that serve as guidance cues for many migratory cell types during injury response (McCaig, 1990; Zhao, 2009; Yamashita, 2013) and limb development (Borgens, 1984; Borgens et al., 1987). Understanding plasma membrane voltage gradients and transepithelial potentials, and their spatiotemporal transitions in vivo, is a key enabling step for the field of developmental bioelectricity and its applications.

Understanding and learning to control patterning signals requires a quantitative appreciation of their intrinsic dynamics and the way they evolve through time. Since the pioneering work of Turing (Turing, 1952; Raspopovic et al., 2014; Watanabe and Kondo, 2015), much effort has gone into mathematical modeling of the dynamics of biochemical signals and their gradients. While there are many platforms for modeling spiking activity in the brain (Bower and Beeman, 2007), there are few available frameworks for formulating predictive models of bioelectric signaling during slower processes involved in somatic cell pattern regulation (Cervera et al., 2016), and even fewer working from the more biorealistic perspective of ionic concentrations and movements, rather than an equivalent electric circuit model. Such biorealistic models are crucial if we are to develop effective interventions that target powerful bioelectric control processes. Furthermore, ion channels and GJs are themselves voltage-sensitive (Nau, 2008; Palacios-Prado and Bukauskas, 2009). This means that cell groups can implement highly non-linear behaviors and feedback loops that are too complex to predict or control by direct inspection. While recent efforts have begun to model some of the interesting behavior of these GJ-coupled dynamical systems (Cervera et al., 2014, 2015; Law and Levin, 2015), there is a need for a flexible, powerful platform to facilitate in silico experimentation and model-building, and for connecting bioelectric dynamics with other aspects of physiology, physical forces, and genetic networks. The availability of a realistic modeling system for bioelectricity will enable (1) formulation of models of specific patterning events based on realistic physiological and channel expression data, (2) design of predicted intervention strategies for inducing desired changes in electrical state and downstream patterning outcomes, and (3) investigation of the broader capabilities of non-neural bioelectrical networks for use in synthetic biology (Doursat and Sanchez, 2014; Kamm and Bashir, 2014; Mustard and Levin, 2014) and unconventional computation architectures (Adamatzky and Jones, 2011; Adamatzky et al., 2012).

To improve understanding of these dynamics and facilitate the development of bioelectric pattern control strategies, Applicant developed the presently disclosed method, a finite volume method multiphysics simulator, which predicts bioelectric patterns and their spatio-temporal dynamics by modeling ion channel, ion transporter, ion pump, and gap junction activity and tracking changes to the fundamental property of ion concentration. The presently disclosed method will enable a deep understanding of local and long-range bioelectrical dynamics in tissues and assist the development of specific interventions to achieve greater control of pattern during morphogenesis and remodeling. Moreover, as a core component of enabling the unraveling of the bioelectrical dynamics of tissues in this exciting emerging field, Applicant has created the presently disclosed method, integrating a diverse range of mechanisms and physiologies to enable model building and hypothesis testing at a level congruent with experimental observables, including electrodiffusion of multiple ions under chemical and electrical gradients in various contexts; consideration of concentration, charge, voltage, and current in both intra- and extracellular networks in order to capture important signals, such as tissue-wide endogenous ion currents, TEP, and local field potentials; and dynamic control of membrane permeability and gap junction state to simulate voltage and ligand-gated channels.

### SUMMARY OF THE PRESENT TECHNOLOGY

In general, one aspect the invention features a system comprising: a computer-readable medium having instructions that when executed cause a processor to generate a differentiable bioelectric tissue simulator for deriving one or more drug, electric field, or gene therapy interventions that produces a predetermined bioelectric state of a tissue, the differentiable bioelectric tissue simulator executing steps comprising: generating, by a processor, a computer model comprising an array corresponding to a set of cells, each cell within the set of cells comprising an intracellular ion concentration, an intracellular chemical substance concentration, a membrane ionic permeability, and a membrane chemical substance permeability, the computer model further comprising an extracellular environment interconnecting the array of cells, the extracellular environment comprising an extracellular ion concentration and an extracellular chemical substance concentration; assigning, by the processor, a value to each of the intracellular ion concentration, the intracellular chemical substance concentration, the membrane ionic permeability, the membrane chemical substance permeability, the extracellular ion concentration, and the extracellular chemical substance concentration; iteratively calculating, by the processor, an intracellular voltage value for at least one cell within the set of cells, wherein, with each iteration, the processor: calculates a membrane voltage; calculates a voltage sensitive membrane permeability; calculates an extracellular ion concentration gradient and a voltage gradient; calculates a membrane- and an extracellular-ion flux over time, t; and updates, by the processor, the intracellular ion concentration and extracellular ion concentration of each of the cells based on the calculated membrane- and extracellular-ion fluxes; and outputting, by the processor, the intracellular voltage of at least one cell.

In some embodiments, the system further comprises a computational graph comprising one or more mathematical operations; a derivative calculated for each of said one or more mathematical operations, which derivative is calculated by an auto-differentiation process; a partial derivative calculated for each of said one or more mathematical operations, which partial derivative is calculated by a chain rule; one or more variables of the mathematical operations selected by the user; and a gradient descent of the partial derivatives to identify required changes in the selected variables to produce the predetermined bioelectric state.

In some embodiments, the one or more variables correspond to ion channel parameters, and the predetermined bioelectric state corresponds to a bistable membrane voltage.

In some embodiments, the predetermined bioelectric state corresponds to the ionic permeability, intracellular ion concentration, extracellular ion concentration, or combinations thereof of cells of a tissue undergoing a biological process, wherein ionic permeability intracellular ion concentration, extracellular ion concentration, or combinations thereof of cells of the tissue undergoing the biological process is determined by imaging, electrophysiological recordings, measurements of gene expression, measurements of mRNA expression, measurements of protein expression, or combinations thereof.

In some embodiments, the predetermined bioelectric state corresponds to the ionic permeability, intracellular ion concentration, extracellular ion concentration, or combinations thereof of cells of a tissue resulting from treatment with one or more drug, electric field, or gene therapy interventions, wherein ionic permeability, intracellular ion concentration, extracellular ion concentration, or combinations thereof of cells of the tissue undergoing the treatment is determined by imaging, electrophysiological recordings, measurements of gene expression, measurements of mRNA expression, measurements of protein expression, or combinations thereof. In some embodiments, the one or more drug, electric field, or gene therapy interventions used to determine the predetermined state is different than the one or more drug, electric field, or gene therapy interventions used to derive the predetermined state.

In some embodiments, the biological process is tissue regeneration.

In some embodiments, the biological process is a restored natural voltage state.

In some embodiments, the biological process is wound healing, tissue and organ engineering, growth of artificial meat, or an alternative immune system response.

In some embodiments, the tissue is selected from the group consisting essentially of an internal organ, a digit, a limb, a muscle, skin, nose, eyes, ears, or brain.

In another aspect, the invention features a method of treating a wound, injury, or congenital disorder in a patient in need thereof, the method comprising contacting the patient with a pharmaceutical composition, electrical field, or gene therapy, wherein the selection and/or placement of the pharmaceutical composition, electrical field, or gene therapy is determined by the presently disclosed system.

In another aspect, the invention features a method of regenerating a tissue or engineering an organ, the method comprising contacting a group of cells with a pharmaceutical composition, electrical field, or gene therapy, wherein the selection and/or placement of the pharmaceutical composition, electrical field, or gene therapy is determined by the presently disclosed system.

In some embodiments, the gene therapy comprises expression of an ion channel corresponding to the ion channel parameters identified by the presently disclosed system.

In some embodiments, a bistable membrane voltage is switched from a depolarized to a hyperpolarized membrane voltage, or from a hyperpolarized to a depolarized membrane voltage.

In another aspect, the invention features a method comprising: generating a computer model comprising an array corresponding to a set of cells, each cell within the set of cells comprising an intracellular ion concentration and a membrane ionic permeability, the computer model further comprising an extracellular environment interconnecting the array of cells, the extracellular environment comprising an extracellular ion concentration; assigning a value to each of the intracellular ion concentration, the membrane ionic permeability, and the extracellular ion concentration; iteratively calculating, by a processor, an intracellular voltage value for at least one cell within the set of cells, wherein, with each iteration, the processor: calculates a membrane voltage; calculates a voltage sensitive membrane permeability; calculates an extracellular ion concentration gradient and a voltage gradient; calculates a membrane ion flux and an extracellular ion flux over time, t; and updates the intracellular ion concentration and the extracellular ion concentration of each of the cells based on the calculated membrane ion flux and extracellular ion flux; and outputting by the processor the intracellular voltage of at least one cell.

In some embodiments, each cell within the array of cells further comprises: an intracellular chemical substance concentration, a membrane chemical substance permeability, and an extracellular chemical substance concentration; wherein the assigned value further comprises: the intracellular chemical substance concentration, the membrane chemical substance permeability, and the extracellular chemical substance concentration; and wherein the iteration further comprises: calculating a gap junction voltage; calculating a voltage sensitive gap junction conductance; calculating a chemical substance sensitive membrane permeability; calculating a chemical substance sensitive gap junction conductance; calculating a concentration growth and/or decay rate of a chemical substance; calculating a forward and/or reverse reaction rate for a chemical reaction; calculating a chemical substance gradient; calculating a gap junction-mediated ion flux over time, t; calculating a membrane-, an extracellular-, a gap junction-mediated chemical substance flux over time, t; updating the intracellular ion concentration and extracellular ion concentration of each of the cells based on the calculated gap junction-mediated ion fluxes; and updating the intracellular chemical substance concentration and extracellular chemical substance concentration of each of the cells based on the calculated membrane-, extracellular-, and gap junction-mediated chemical substance fluxes.

In some embodiments, the ion flux or chemical substance flux is calculated using a GHK or Nernst Planck flux equation.

In some embodiments, at least one of the intracellular ion concentrations, extracellular ion concentration, ionic permeabilities, intracellular chemical substance concentrations, extracellular chemical substance concentration, ionic permeabilities, chemical substance permeabilities, voltage dependent permeabilities, or gap junction permeabilities, is received by the processor.

In some embodiments, the cell comprises a perimeter having multiple facets, wherein a facet of the perimeter is opposed by one or more geometrically equivalent facet(s) of an adjacent cell and/or an adjacent boundary of the array, wherein the boundary of the array comprises a large extracellular space, wherein optionally the perimeters of a 2D array of cells each comprises a hexagonal planar geometry.

In some embodiments, a facet of the perimeter comprises a small extracellular space shared with an adjacent facet.

In some embodiments, the cell further comprises a plurality of sub compartments, wherein the processor assigns an intracellular ion concentration to the sub compartment, and wherein the iteration further comprises calculating a Nernst Planck flux equation between any two sub compartments.

In some embodiments, outputting comprises at least one of displaying a number for an intracellular voltage for at least one cell, displaying a graph comprising an intracellular voltage for at least one cell, or displaying an intracellular voltage over time, T.

In some embodiments, the array of cells is 2D or 3D.

In some embodiments, the array of cells, ionic permeability, chemical substance permeability, intracellular ion concentration, extracellular ion concentration, extracellular chemical substance concentration, intracellular chemical substance concentration or combinations thereof corresponds to an injured or an uninjured tissue, and wherein the array of cells, ionic permeability, intracellular ion concentration, extracellular ion concentration, extracellular chemical substance concentration, or intracellular chemical substance concentration or combinations thereof of cells of the tissue is determined by imaging, electrophysiological recordings, measurements of gene expression, or combinations thereof.

In some embodiments, the tissue is selected from the group consisting essentially of an internal organ, a digit, a limb, a muscle, skin, nose, eyes, ears, and brain.

In some embodiments, the ionic permeability of at least one cell is modified by the pharmacological properties of one or more drugs, an electric field, or a gene therapy.

In another aspect, the invention features a system comprising: a computer-readable medium having instructions that when executed cause a processor to generate a differentiable bioelectric tissue simulator for deriving one or more drug, electric field, or gene therapy interventions that produces a predetermined bioelectric state of a tissue, the differentiable bioelectric tissue simulator executing steps comprising: generating, by a processor, a computer model comprising an array corresponding to a set of cells, each cell within the set of cells comprising an intracellular ion concentration, an intracellular chemical substance concentration, a membrane ionic permeability, and a membrane chemical substance permeability, the computer model further comprising an extracellular environment interconnecting the array of cells, the extracellular environment comprising an extracellular ion concentration and an extracellular chemical substance concentration; assigning, by the processor, a kinetic state and transition rate to each of the membrane ionic and chemical substance permeabilities; iteratively calculating, by the processor, a change in ion and chemical substance concentration over time, t, wherein, with each iteration, the processor: calculates a membrane potential; calculates an electric field; updates, by the processor, the kinetic transition rate and state by the calculated ion and chemical substance concentration, membrane potential, and electric field; and outputs, by the processor, an intracellular voltage, ion concentration, and/or chemical substance concentration for at least one cell.

In some embodiments, the membrane permeability corresponds to the permeability of a voltage or ligand gated ion channel, a gap junction, and/or an ion or chemical substance transporter. In some embodiments, the kinetic transition rate is voltage or concentration dependent.

In some embodiments, the kinetic state and transition rate for each of the membrane ionic and chemical substance permeabilities is updated simultaneously.

In another aspect, in some embodiment the system further comprises: a computational graph comprising one or more mathematical operations; a derivative calculated for each of said one or more mathematical operations, which derivative is calculated by an auto-differentiation process; a partial derivative calculated for each of said one or more mathematical operations, which partial derivative is calculated by a chain rule; one or more variables of the mathematical operations selected by the user; and a gradient descent of the partial derivatives to identify required changes in the selected variables to produce the predetermined bioelectric state.

In some embodiments, the one or more variables correspond to ion channel parameters, and wherein the predetermined bioelectric state corresponds to a bistable membrane voltage.

In some embodiments, the predetermined bioelectric state corresponds to the ionic permeability, chemical substance permeability, intracellular ion concentration, extracellular ion concentration, extracellular chemical substance concentration, intracellular chemical substance concentration, or combinations thereof of cells of a tissue undergoing a biological process, wherein ionic permeability, chemical substance permeability, intracellular ion concentration, extracellular ion concentration, extracellular chemical substance concentration, intracellular chemical substance concentration or combinations thereof of cells of the tissue undergoing the biological process is determined by imaging, electrophysiological recordings, measurements of gene expression, measurements of mRNA expression, measurements of protein expression, or combinations thereof.

In some embodiments, the predetermined bioelectric state corresponds to the ionic permeability, chemical substance permeability, intracellular ion concentration, extracellular ion concentration, extracellular chemical substance concentration, intracellular chemical substance concentration, or combinations thereof of cells of a tissue resulting from treatment with one or more drug, electric field, or gene therapy interventions, wherein ionic permeability, chemical substance permeability, intracellular ion concentration, extracellular ion concentration, extracellular chemical substance concentration, intracellular chemical substance concentration, or combinations thereof of cells of the tissue undergoing the treatment is determined by imaging, electrophysiological recordings, measurements of gene expression, measurements of mRNA expression, measurements of protein expression, or combinations thereof.

In some embodiments, the one or more drug, electric field, or gene therapy interventions used to determine the predetermined state is different than the one or more drug, electric field, or gene therapy interventions used to derive the predetermined state.

In some embodiments, the biological process is tissue regeneration.

In some embodiments, the biological process is a restored natural voltage state.

In some embodiments, the biological process is wound healing, tissue and organ engineering, growth of artificial meat, or an alternative immune system response.

In some embodiments, the tissue is selected from the group consisting essentially of an internal organ, a digit, a limb, a muscle, skin, nose, eyes, ears, or brain.

In another aspect, the invention features a method of treating a wound, injury, or congenital disorder in a patient in need thereof, the method comprising contacting the patient with a pharmaceutical composition, electrical field, or gene therapy, wherein the selection and/or placement of the pharmaceutical composition, electrical field, or gene therapy is determined by the presently disclosed system.

In another aspect, the invention features a method of regenerating a tissue or engineering an organ, the method comprising contacting a group of cells with a pharmaceutical composition, electrical field, or gene therapy, wherein the selection and/or placement of the pharmaceutical composition, electrical field, or gene therapy is determined by the presently disclosed system.

In some embodiments, the gene therapy comprises expression of an ion channel corresponding to the ion channel parameters identified by the presently disclosed system.

In some embodiments, a bistable membrane voltage is switched from a depolarized to a hyperpolarized membrane voltage, or from a hyperpolarized to a depolarized membrane voltage.

In another aspect, the invention features a method comprising: generating a computer model comprising an array corresponding to a set of cells, each cell within the set of cells comprising an intracellular ion concentration and a membrane ionic permeability, the computer model further comprising an extracellular environment interconnecting the array of cells, the extracellular environment comprising an extracellular ion concentration; assigning a kinetic state and transition rate to the membrane ionic permeability; iteratively calculating, by a processor, a change in ion concentration over time, t, wherein, with each iteration, the processor: calculates a membrane potential; calculates an electric field; updates, by the processor, the kinetic transition rate and state by the calculated ion concentration, membrane potential, and electric field; and outputs, by the processor, an intracellular voltage and/or ion concentration, for at least one cell.

In some embodiments, each cell within the array of cells further comprises: an intracellular chemical substance concentration, a membrane chemical substance permeability, and an extracellular chemical substance concentration; wherein the assigning further comprises: assigning a kinetic state and transition rate to the chemical substance permeability; and wherein the iteration further comprises: calculating a change in chemical substance concentration over time, t; updating, by the processor, the kinetic transition rate and state by the calculated chemical substance concentration; and outputting, by the processor, a chemical substance concentration for at least one cell.

In some embodiments, the membrane ion permeability or chemical substance permeability is calculated using a GHK or Nernst Planck flux equation.

In some embodiments, at least one of the intracellular ion concentrations, extracellular ion concentration, ionic permeabilities, intracellular chemical substance concentrations, extracellular chemical substance concentration, or chemical substance permeabilities, is received by the processor.

In some embodiments, the cell comprises a perimeter having multiple facets, wherein a facet of the perimeter is opposed by one or more geometrically equivalent facet(s) of an adjacent cell and/or an adjacent boundary of the array, wherein the boundary of the array comprises a large extracellular space, wherein optionally the perimeters of a 2D array of cells each comprises a hexagonal planar geometry.

In some embodiments, a facet of the perimeter comprises a small extracellular space shared with an adjacent facet.

In some embodiments, the cell further comprises a plurality of sub compartments, wherein the processor assigns an intracellular ion concentration to the sub compartment, and wherein the iteration further comprises calculating a Nernst Planck flux equation between any two sub compartments.

In some embodiments, outputting comprises at least one of displaying a number for an intracellular voltage for at least one cell, displaying a graph comprising an intracellular voltage for at least one cell, or displaying an intracellular voltage over time, T.

In some embodiments, the array of cells is 2D or 3D.

In some embodiments, the array of cells, ionic permeability, chemical substance permeability, intracellular ion concentration, extracellular ion concentration, extracellular chemical substance concentration, intracellular chemical substance concentration or combinations thereof corresponds to an injured or an uninjured tissue, and wherein the array of cells, ionic permeability, intracellular ion concentration, extracellular ion concentration, extracellular chemical substance concentration, or intracellular chemical substance concentration or combinations thereof of cells of the tissue is determined by imaging, electrophysiological recordings, measurements of gene expression, or combinations thereof.

In some embodiments, the tissue is selected from the group consisting essentially of an internal organ, a digit, a limb, a muscle, skin, nose, eyes, ears, and brain.

In some embodiments, the ionic permeability of at least one cell is modified by the pharmacological properties of one or more drugs, an electric field, or a gene therapy.

In another aspect, the invention features an in silico network of virtual cells comprising a digital tissue model represented as a differentiable reaction-diffusion kinetic network, said digital tissue model having at least one bistable virtual cell bioelectrically coupled to a plurality of interconnected virtual cells and virtual environmental compartments, wherein the at least one bistable virtual cell exhibits a membrane ionic permeability that is associated with an artificial ion channel, said digital tissue model being further characterized as capable of propagating a voltage change across the plurality of interconnected virtual cells when a state of the at least one bistable virtual cell is switched from a depolarized state to a hyperpolarized state or vice versa.

In some embodiments, the status of the at least one bistable virtual cell is switched in response to a virtual action of a drug, an electric field, or both.

In some embodiments, the drug, electric field, or both is selected to propagate a voltage change that is exhibited by, observed in, or demonstrated in a living tissue.

In some embodiments, the in silico network is a digital twin of a living tissue, such that the cell geometry and an ionic membrane permeability are derived from living tissue.

In another aspect, the invention features a method of identifying molecular bioelectric targets for potential bioelectric manipulation or electroceutical intervention comprising interrogating an in silico network of virtual cells, which network comprises a digital twin of a living tissue, by conducting bioelectric simulations in combination with a search algorithm, which simulations iteratively fit and optimize a given bioelectric state to match a predefined bioelectric state under conditions that reveal molecular bioelectric targets that are capable of influencing, restoring, or redirecting bioelectric signaling, thereby enabling cellular decision-making.

In some embodiments, a bioelectric state includes spatial distributions of bioelectric patterns, temporal dynamics of bioelectric signaling, or a combination of both.

In some embodiments, the spatial distributions of bioelectric patterns comprise voltage gradients, ion concentration gradients, pH gradients, electrochemical field patterns, gap junctions' connectivity patterns, regional expression, local distribution of ion channels and pumps, or combinations thereof.

In some embodiments, the temporal dynamics of bioelectric signaling comprise oscillatory signals, transient signals, sustained or steady state changes, signal propagation dynamics, propagation speed, signal duration, spiking patterns, non-periodic spikes or bursts of bioelectric activity, synchronization patterns, phase relationships between cells, patterns of signal initiation or termination, or combinations thereof.

In some embodiments, the given bioelectric state comprises a diseased or pathological condition.

In some embodiments, the predefined bioelectric state comprises a normal or healthy bioelectric state.

In some embodiments, any identified molecular bioelectric targets are ranked according to their inferred capacity to functionally generate and maintain a predetermine bioelectric profile within the predefined state.

In some embodiments, bioelectric manipulation comprises electrical stimulation.

In some embodiments, electroceutical intervention comprises an exposure to a compound or a combination of compounds.

In some embodiments, a compound is selected from the group consisting of a chemical compound, a small molecule, a biologic, a polypeptide, an antibody, a single-stranded or double-stranded nucleic acid, cell-based therapies, nanoparticles, optogenetics, or combinations thereof.

In some embodiments, the bioelectric manipulation or electroceutical intervention modulates an intrinsic, molecular, bioelectric component of biological tissue.

In some embodiments, the method enables targeted bioelectric control of tissue function, tissue repair, organ repair, disease outcome, or combinations thereof.

The following detailed description is exemplary and explanatory and is intended to provide further explanation of the invention.

### BRIEF DESCRIPTION OF THE DRAWINGS

FIG. 1: The core processing step for iteratively updating electrophysiological properties of each cell of an array of cells is shown. In a hexagonal planar geometry, each cell of the array of cells will complete an iteration with six neighboring cells and associated extracellular spaces.

FIG. 2: Additional processing steps for inclusion of gap junctions are shown.

FIG. 3: An exemplary model of 13 hexagonal planar cells and 7 large extracellular spaces in a networked array is shown.

FIG. 4: An exemplary computer environment that can be used to provide a network-based implementation of the methods and processes is shown.

FIG. 5: An overview of backpropagation for automatic differentiation (AD) is shown.

FIG. 6: A bistable Kir2.1 ion channel is shown. Current vs voltage relationships are used to infer the stability of a voltage state. Where the black line (denoting the current-voltage relationship) crosses the y=0 line, a critical point is present. These points can be stable (red) or unstable (blue). The presence of two stable critical points define bistability. On the left is the relationship produced by the parameters from the Channelpedia model, where only a single stable point is present. On the right is the relationship identified by using gradient descent in the simulator.

FIG. 7: Bistability of cell potentials is shown. Both cells were initially given a hyperpolarizing current to reduce their resting potential to −80 mV. At t=1 seconds, Cell 1 is depolarized, which inactivates the Kir channel, causing its resting potential to become approximately 0 mV.

FIG. 8: Loss vs number of iterations is shown. Loss is the average squared difference between the measured voltage values and the target voltage values. The discontinuity that appears at iteration 20 indicates that a “supercritical pitchfork bifurcation” has occurred, where the single stable critical point has split into two stable and one unstable critical points. This immediately satisfies the goal of having two stable states, and as a result the loss rapidly decreased.

FIG. 9: Alternative, preferred, processing steps for iteratively calculating and updating electrophysiological properties of each cell of an array of cells using a kinetic scheme is shown. In a hexagonal planar geometry, each cell of the array of cells will complete an iteration with six neighboring cells and associated extracellular spaces.

FIG. 10: Additional processing steps for the kinetic solver are shown.

FIG. 11: Kinetic states and transition rates of a sodium calcium exchanger are shown in network form. Steps with the “Bind” suffix indicate ion binding rates, while steps with “Unbind2 indicate ion dissociation. The suffixes “io” and “oi” signify translocation across the membrane.

### DETAILED DESCRIPTION

It is to be appreciated that certain aspects, modes, embodiments, variations and features of the present methods are described below in various levels of detail in order to provide a substantial understanding of the present technology.

### Definitions

Unless defined otherwise, all technical and scientific terms used herein have the same meanings as commonly understood by one of ordinary skill in the art to which this disclosure belongs. All technical and patent publications cited herein are incorporated herein by reference in their entirety. Nothing herein is to be construed as an admission that the disclosure is not entitled to antedate such disclosure by virtue of prior disclosure.

As used in the specification and claims, the singular form “a,” “an” and “the” include plural references unless the context clearly dictates otherwise. For example, the term “a polypeptide” includes a plurality of polypeptides, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions disclosed herein. Embodiments defined by each of these transition terms are within the scope of this disclosure.

As used herein, the term “optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

The term “substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

As used herein, an “array of cells” refers to 2, 3, 4, 5, 6, 7, 8, 9, 10, 100, 1000, or more cells arranged in a 2D or 3D space. In some embodiments, cells are equidistant from each other and network to 6 cells. In other embodiments, cells are spaced at various distances from each other and directly network to 2, 3, 4, 5, 6, 7, 8, 9, 10 or more cells.

As used herein, “directly networked” refers to cells directly influencing the electrophysiological properties of an adjacent cell without an extracellular environment intermediary (e.g., a GJ or a synapse).

As used herein, “indirectly networked” refers to cells indirectly influencing the electrophysiological properties of an adjacent cell through an extracellular environment intermediary (e.g., local field effects).

As used herein, an “extracellular environment” refers to the environment outside of a cell's perimeter. In some embodiments, 6 identical extracellular environments are associated with a cell indirectly networked to 6 equidistant cells. In other embodiments, 1, 2, 3, 4, 5, 6, 7, 8, 9, 10 or more distinct extracellular environments are associated with a cell directly and indirectly networked to 1, 2, 3, 4, 5, 6, 7, 8, 9, 10 or more cells spaced at various distance from each other. In any and all embodiments, an extracellular environment may be interconnected with an adjacent extracellular environment, allowing for the flow of ions between extracellular environments.

As used herein, “iteratively calculating” refers to calculating electrophysiological equations for a timestep t, updating variables associated with the calculation, and then calculating electrophysiological equations for another timestep/using the updated variables. In some embodiments, “T” refers to the duration of a simulation.

As used herein, “voltage sensitive membrane permeability” refers to membrane permeability affected by voltage, including but not limited to permeability caused by a voltage-sensitive ion channel.

As used herein, “electroosmotic flow” refers to the motion of liquid induced by an applied potential across two or more extracellular environments or two or more intracellular environments.

As used herein, “spatially variable diffusion constant” refers to diffusion constants that vary in space, e.g., a person skilled in the art understands that the diffusion constant of an open extracellular space will be larger than a GJ or tight junction.

As used herein, “membrane characteristics” refers to properties of the membrane, including but not limited to permeability, capacitance, thickness, and surface area.

As used herein, “ion flux” refers to the net movement of ions from one compartment to another, including but not limited to ion flux from an extracellular environment to an intracellular space, or ion flux from one compartment of a cell to another compartment of the cell.

As used herein, “boundaries” refers to facets of the geometric perimeter of a cell. In some embodiments, a cell is networked to six equidistant cells, forming a hexagonal geometric perimeter, comprising 6 boundary facets.

As used herein, “gene therapy” refers to any treatment that modifies or manipulates the expression of genetic material to alter the biological properties of a living cells. Genetic material includes, for example, DNA, RNA, mRNA, or equivalents from any source, whether natural occurring or artificially synthesized.

As used herein, “drug” refers to any substance that may affect the electrophysiological properties of a cell, the effects of which can be modeled or simulated as disclosed herein. For example, a drug may alter the excitability of membranes, act as agonists or antagonists for specific ion channels or pumps, block an ion channel or pump, alter synaptic vesicle cycling and fusion, alter the clearance of signaling molecules from a synaptic cleft or extracellular space, cause the formation of membrane pores, alter mitochondrial function, alter protein expression or degradation, alter the phosphorylation status of a protein, or act by any other mechanism that results in altered electrophysiological properties of a cell.

As used herein, “chemical substance” refers a nucleic acid, protein, chemical, neurotransmitter, or signaling molecule produced by a cell or exogenously applied to a cell, and includes precursors, intermediaries, and fragments thereof.

As used herein, “virtual” refers to digital information carried out, accessed, or stored by means of a computer, especially over a network.

### Model Overview

Voltages (electric potential energies) are created by net electrical charge. In typical electrical systems, such as metals and semiconductors, the charge carriers are electrons or the absence of electrons (holes). In electrolytes, ions from dissolved salts can develop concentration profiles generating net charge in a region of space and, therefore, create voltages. Furthermore, mass flux of ions can generate a net current, which is associated with intracellular and tissue-wide electric fields. Therefore, ions are the fundamental units of the bioelectrical system, and their concentrations, mass fluxes, and transport mechanisms are ultimately important. The presently disclosed method can consider ions relevant to most living systems: Na⁺, K⁺, Cl⁻, Ca²⁺, HCO₃⁻, H⁺, and charged macromolecules, such as proteins (X). In addition, the presently disclosed method can consider the movement of a charged biomolecule, such as a voltage reporter dye, glutamate, serotonin or inositol triphosphate (symbolized as Yⁿ⁻ or Yⁿ⁺, where n is a variable charge number).

Cells create and control Vₘₑₘ by selectively altering ion fluxes across their membrane. Ion pumps, such as the sodium potassium pump (Na/K-ATPase), use free-energy released from ATP hydrolysis to move ions across the insulating cell membrane, creating net ionic charge density and voltage gradients inside and outside of the cell, similar to a self-charging capacitor (Veech et al., 1995). Ion channels in the plasma membrane allow charge to move under these concentration and voltage gradients, altering charge densities and thereby changing the concentration and voltage gradients to create bioelectrical signals. The presently disclosed method keeps track of ion concentrations and ion fluxes in space and time, reducing them to net charge distributions inside and outside of the cell, using these net charges to calculate voltages inside and outside of the cellular space, calculating changes to concentrations resulting from ion mass fluxes resulting from concentration/voltage gradients and by active ion pumps, and calculating endogenous currents from the net mass flux of ions. Membrane permeability to specific ions is used as a dynamic variable to simulate the action of specific ion channels (including K⁺ leak channels, calcium gated K⁺ and Cl⁻ channels, and voltage-gated Na⁺, K⁺, and Ca²⁺ channels).

TABLE 1
summarizes key parameters and their units:

| Name | Location | Units |
|---|---|---|
| Characteristic data | | |
| Cell position x, y, z | Cell | m |
| Cell volume | Cell | m³ |
| Cell surface area | Cell | m² |
| Gap junction type | Cell | — |
| Baseline gap junction diffusion rate | Cell | $\frac{{m}^{2}}{s}$ |
| Cell chemical reactions | Cell | — |
| Membrane position | Membrane | m |
| Membrane capacitance | Membrane | $\frac{F}{{m}^{2}}$ |
| Membrane width, height, thickness | Membrane | m |
| Baseline ionic diffusion rates | Membrane | $\frac{{m}^{2}}{s}$ |
| Membrane flux mechanisms | Membrane | — |
| Environment position x, y, z | Environment | m |
| Environment volume | Environment | m³ |
| Environment chemical reactions | Environment | — |
| Environment diffusion scaling | Environment | $\frac{{m}^{2}}{s}$ |
| Temperature | All | K |
| Network data | | |
| Cell to cell network | | |
| Gap junction network | | |
| Cell to membrane network | | |
| Membrane to environment network | | |
| Cell to environment network | | |
| Environment to environment network | | |
| Kinetic interaction network | | |
| Simulation variables/outputs | | |
| Ion concentrations | Cell, environment | $\frac{mM}{L}$ |
| Ion flux | All | $\frac{mM}{{m}^{2} s}$ |
| Chemical substance concentration | Cell, environment | $\frac{mM}{L}$ |
| Chemical substance flux | All | $\frac{mM}{{m}^{2} s}$ |
| Net charge density | Cell, environment | C/m³ |
| Membrane potential | Membrane | V |
| Electric field | Cell, environment | $\frac{V}{m}$ |
| Kinetic state values | All | — |
| pH | Cell, environment | — |

### Core Mathematical Strategy

Biological tissue represents a challenging modeling scenario due to its highly heterogeneous nature, where closely spaced (˜10-30 nm), membrane bound, electrolyte-filled cells are individually interacting with a small extracellular space at individual plasma membranes, and where the extracellular spaces connect with a continuous, aqueous environment at the cell cluster boundary. Individual cells are also connected internally via transmembrane channels, such as gap junctions (GJ), which enable passage of small molecules and ionic current between cells. To manage this involved biophysical situation, the presently disclosed method uses a highly configurable cell array to model the heterogeneous nature of tissues, while also allowing modeling of a continuous environmental space around each cell.

Each modeled cell in the array has a center point, where scalar cell properties, such as ionic concentration and intracellular voltage are defined. Each cell also has a volume and a geometric perimeter, the shape of which is defined by the cell's spatial relationship to proximal cells in the array. In some embodiments, a cell will be directly networked to 6 proximal cells, thereby creating a hexagonal planar cell perimeter geometry. In other embodiments, the cell geometry is any polygonal shape achievable by a cell segmentation algorithm. The volumes of the cell perimeters control the presence or absence of an intracellular space between any two cells, and the volume of that intracellular space. Intracellular spaces also are networked to adjacent intracellular spaces.

In one aspect, electrophysiological equations are calculated for each facet of the geometric perimeter. For example, a cell with hexagonal planar geometry has 6 facets, resulting in the computation of at least 6 sets of electrophysiology equations associated with the cell. In another aspect, a small extracellular space associated with two opposing cell perimeter facets may interconnect with 1, 2, 3, 4, 5, or more other small extracellular spaces, requiring calculation of an equivalent number of sets of electrophysiological equations associated with the small extracellular space. In another aspect, the perimeter of a cell may be adjacent to one or more large extracellular space at the boundary of the cellular array.

The core mathematical operators of differential equations used in the presently disclosed method are gradient, divergence, and Laplacian/inverse Laplacian. Discrete versions of gradient, divergence, and Laplacian/inverse Laplacian are defined, using standard finite difference and finite volume techniques (Schafer, 2006), on the cells and perimeters. These core mathematical operators are then used where required in specific differential equation expressions. Additional information can be found in Reference 1, which is hereby incorporated by reference in its entirety.

### Bio-Electrochemical Mass Transport

Ion transport in bioelectrical systems is influenced by gradients of both concentration and voltage, with ions passively moving by a process known as electrodiffusion, which is a combination of regular diffusion and electrophoretic transport. In general, electrodiffusion is described by the Nernst-Planck differential equation, describing the rate of change in the concentration of an ion:

$$\frac{{dc}_{i}}{dt} = ∇ \cdot ( {D}_{i} ∇ {c}_{i} + \frac{{D}_{i} {z}_{i} q}{{k}_{b} T} ∇ V - \vec{u} {c}_{i} ) . \qquad Equation 1$$

The equation for electrodiffusion can be rewritten as a transition between the kinetic states. See infra, kinetic implementations of the present disclosure.

Ions are actively transported by pumps in the cell membrane. Ions are also actively or passively transported by transporters in the cell membrane. Both passive and active transport processes generate ion fluxes. These combined fluxes can lead to changes in concentration and charge density, and can generate a system-wide ionic current density.

The presently disclosed method assumes passive electrodiffusive mass transport in a multicellular cluster follows two distinct pathways: (1) transmembrane, via intra- and extracellular spaces across the plasma membrane and via intercellular spaces of adjacent cells across gap junctions; (2) between adjacent intra- or extracellular spaces not separated by a membrane. Active transport from ion pumps is always assumed to be transmembrane. Therefore, the presently disclosed method considers the following sources of ion flux:

Transmembrane, from passive electrodiffusive transport resulting from gradients between the local intra- and extracellular spaces and between adjacent cells connected by gap junctions using the Goldman-Hodgkin-Katz Flux equation (GHK Flux equation), which is derived from the Nernst-Planck Differential equation for the case of electrodiffusion across a cell membrane for a non-steady-state Vₘₑₘ (Bowman and Baglioni, 1984):

$${Φ}_{{mem}_{i}} = \frac{{z}_{i} {V}_{mem} {FD}_{i}}{{RTd}_{mem}} ( \frac{{c}_{{cell}_{i}} - {c}_{{env}_{i}} exp ( \frac{- {z}_{i} {V}_{mem} F}{RT} )}{1 - exp ( \frac{- {z}_{i} {V}_{mem} F}{RT} )} ) . \qquad Equation 2$$

Transmembrane, from active transport resulting from ion pump activity:

$${Φ}_{{pump}_{i}} = α ( {c}_{{cell}_{i}} , {c}_{{env}_{i}} , {V}_{mem} , t ) . \qquad Equation 3$$

Intercellular, from passive electrodiffusive transport resulting from gradients between neighboring, GJ networked cells:

$${Φ}_{GJi} = - \frac{{z}_{i} {V}_{G J} {FD}_{{GJ}_{i}}}{{RTd}_{GJ}} ( \frac{{c}_{cellx} - {c}_{{cell}_{y}} exp ( - \frac{{z}_{i} {V}_{G J} F}{R T} )}{1 - exp ( - \frac{{z}_{i} {V}_{G J} F}{R T} )} ) . \qquad Equation 4$$

Φ_{GJi} is the flux of ion i across the gap junction. V_{Gj} is the difference between the cell potentials on either side of the gap junction, D_{Gji} is the gap junction diffusion rate for ion i, and d_{GJ} is the length of the gap junction. Extracellular, from passive electrodiffusive transport resulting from gradients between neighboring environmental spaces:

$${\vec{Φ}}_{{env}_{i}} = - {D}_{i} ∇ {c}_{{env}_{i}} - \frac{{D}_{i} {z}_{i} q}{{k}_{h} T} {c}_{{env}_{i}} ∇ {V}_{env} + {\hat{u}}_{env} {c}_{{env}_{i}} . \qquad Equation 5$$

Changes in concentration are made by assuming the concentration change in an ion depends on the divergence of the net sum of all fluxes of the ion entering or changing in a particular region of the space (i.e., cells or environment):

$$\frac{\partial {c}_{i}}{\partial t} = - ∇ \cdot {Φ}_{{tot}_{i}} . \qquad Equation 6$$

Net ionic charge density was calculated by summing all ion concentrations at a region of space:

$${ρ}_{e} = {\sum }_{i} {Fz}_{i} {c}_{i} . \qquad Equation 7$$

The dynamics of ionic charge density were calculated from the mass flux of all ions:

$$\frac{\partial {ρ}_{e}}{\partial t} = {\sum }_{i} {Fz}_{i} {Φ}_{i} . \qquad Equation 8$$

The total current density of the environment or cell was calculated using the continuity equation in combination with the assumption of bulk electro-neutrality for electrolytes due to charge screening. Using the Continuity equation for current, the current density in a region follows:

$$∇ \cdot {\vec{J}}_{o} + \frac{\partial {ρ}_{e}}{\partial t} = 0. \qquad Equation 9$$

As electro-neutrality (zero net charge density) should be preserved in the bulk electrolyte, the base current density calculated by the presently disclosed method was corrected by assuming that an internal electric field develops in the bulk electrolyte as a result of charge screening, which is the negative gradient of an electric potential:

$$\vec{J} = {J}_{o} - ∇ {ψ}_{i n t} . \qquad Equation 10$$

Substituting equation (10) into equation (9) and rearranging to solve for the internal electric potential:

$${ψ}_{i n t} = {∇}^{- 2} ( ∇ \cdot {\vec{J}}_{o} + \frac{\partial {ρ}_{e}}{\partial t} ) . \qquad Equation 11$$

After obtaining φᵢₙₜ, it is used with equation (10) to produce the corrected current density for the system. Current density in the environment and in cell spaces was treated as separate.

Note that as movement in both concentration and electrical gradients can occur, the transport properties of bioelectrical systems cannot be strictly reduced to electrical constants, such as resistance or conductance. However, examining the Nernst-Planck equation (e.g., equation (1)) reveals that the diffusion coefficient is able to serve as the constant of proportionality for movement in both chemical and electrical terms. In the absence of a concentration gradient, and multiplying by Fz to convert mass flux to ionic current density, the Nernst-Planck Flux equation reduces to:

$${\vec{J}}_{i} = - \frac{{FD}_{i} {z}^{2} q}{{k}_{b} T} {c}_{i} ∇ V . \qquad Equation 12$$

Noting the definition of an electric field, equation (12) parallels the equation relating current density to electric field via media conductivity:

$$\vec{J} = \frac{1}{γ} \vec{E} . \qquad Equation 13$$

Therefore, the presently disclosed method makes use of diffusion constants to characterize ion transport in different regions of the multicellular cluster, but can approximate conversions between conductivity and the diffusion constant.

### Biological Voltages

To calculate potentials Vᵢₙₜᵣₐ and Vₑₓₜᵣₐ, first the net charge density ρₑ in each cell or environment section is calculated using equation (7) by summing all the ion concentrations and multiply by Fz. This sum is converted to a surface charge density by multiplying by the divergence scaling factor (cell (or environment) volume÷cell (or environment) surface area). From this, the cell (or environment) potential is derived by multiplying the cell (or environment) surface charge density by 1/cell (or environment) capacitance. Cell and environment capacitances are user specified variables, that depend on the tissue being modelled.

### Ion Pumps

Ion pumps were modeled as enzymes using standard Michaelis-Menten enzyme kinetic relations, with reaction rates determined by thermodynamic arguments.

The equilibrium constant of a reaction, K_{eqm}, can be expressed both in terms of the reaction free energy under standard conditions, and in terms of the reaction's product concentrations (index k) and those of its reactants (index j) where aₖ and aⱼ represent coefficients of stoichiometry for the reaction (Beard and Qian, 2007; Pekar, 2015):

$${K}_{eqm} = exp ( - \frac{Δ {G}_{react}^{eqm}}{RT} ) = \frac{\sum {c}_{k}^{{a}_{k}}}{\sum {c}_{j}^{{a}_{j}}} . \qquad Equation 14$$

The electrochemical potential of a substance at concentration cᵢ with charge zᵢ in a region where there is a voltage V is expressed:

$${μ}_{i} = {μ}_{o} + RT ln ( {c}_{i} ) + {z}_{i} FV . \qquad Equation 15$$

Furthermore, the overall free-energy of a reaction is described as the sum of the (electro) chemical potentials of its products (index k) minus those of its reactants (index)) where aₖ and aⱼ represent coefficients of stoichiometry for the reaction:

$$Δ {G}_{reaction} = \sum {a}_{k} {μ}_{k} - \sum {a}_{j} {μ}_{j} . \qquad Equation 16$$

Using the Na/K-ATPase pump as an example, the overall reaction for the Na/K-ATPase pump is:

$$3 {c}_{Na}^{i n} + 2 {c}_{K}^{out} + ATP ↔ 3 {c}_{Na}^{out} + 2 {c}_{K}^{i n} + ADP + P . \qquad Equation 17$$

From the abovementioned fundamental chemical principals, the overall free energy, ΔGₚᵤₘₚ, for the Na/K-ATPase pump reaction can be expressed (Smith and Crampin, 2004):

$$\begin{matrix}Δ {G}_{pump} = Δ {G}_{ATP}^{o} + RT ln ( Ω ) - {FV}_{mem} \\ Ω = \frac{cADP cP cN {a}_{out}^{3} {cK}_{in}^{2}}{cATP cN {a}_{out}^{3} {cK}_{in}^{2}}\end{matrix} . \qquad Equation 18$$

when ΔGₚᵤₘₚ=0, the reaction is at equilibrium. Using equation (23), an expression for the Na/K-ATPase pump reaction equilibrium constant in terms of the standard free energy for ATP hydrolysis and cell Vₘₑₘ is:

$${K}_{NaKATP}^{eqm} = exp ( \frac{- {ΔG}_{ATP}^{o} + {FV}_{mem}}{RT} ) . \qquad Equation 19$$

Following with basic Michaelis-Menten enzyme kinetics, an estimate for the rate of the reversible enzymatic pump reaction follows as:

$$α NaKATP - {α}_{o} ( \frac{\frac{cATP}{{K}_{ATP}} \frac{{cNa}_{in}}{{K}_{Na}} \frac{{cK}_{out}}{{K}_{K}}}{( 1 + \frac{cATP}{{K}_{ATP}} ) ( 1 + \frac{{cNa}_{in}}{{K}_{Na}} ) ( 1 + \frac{{cK}_{out}}{{K}_{K}} )} ) \times ( 1 - \frac{Ω}{{K}_{NaKATP}^{eqm}} ) . \qquad Equation 20$$

In addition to Na/K-ATPase pumps, the presently disclosed method can optionally simulate Ca-ATPase, H/K-ATPase, and V-ATPase pumps using free-energy regulated pumping rates analogous to that outlined above for the Na/K-ATPase pump.

### Voltage-Gated Channels

A range of voltage-gated channel types have been implemented in the presently disclosed method using Hodgkin-Huxley style differential equations to define the state of membrane diffusion to a specific ion (e.g., Na⁺) as a function of Vₘₑₘ and time. Specific parameters and functional relations were obtained from the online database, Channelpedia (Ranjan et al., 2011).

In some aspects, the disclosed method uses a combined generic voltage-gated sodium channel (NaV) from (Hamill et al., 1991), and a delayed-rectifier voltage-gated potassium channel (KV1.2) from (Sprunger et al., 1996), to generate excitable signals. Any voltage-gated ion channel may be used. A standard Hodgkin-Huxley style model uses an electrical equivalent circuit equation to determine changes to current and voltage across a membrane, with a set of differential equations controlling the conductance of the membrane (Nelson, 2004). Since conductance is proportional to the membrane diffusion constant for a particular ion (see equations (12) and (13)), the present disclosure uses the same Hodgkin-Huxley style equations developed to describe membrane conductivity state to describe the membrane diffusion state of a particular ion, updating subsequent changes to currents and voltages using its own methods, as described in the above. Details regarding voltage-gated channel dynamics are specified in (Pietak & Levin, 2016), hereby incorporated by reference.

### Gap Junctions

Gap junctions were modeled as (optionally) voltage-sensitive conduits influencing the intercellular diffusion coefficient for all ions via diffusion-constant scaling factors. Simulated transport through GJ used the Nernst-Planck equation (equation (1)) to update concentration of all ions moving under intercellular concentration and voltage gradients. In the absence of GJ, cells were modeled to have an intercellular diffusion coefficient of zero.

Voltage gating of GJ was described using the kinetic model of (Harris et al., 1983), which calculates GJ open/closed state (β_{GJ}) dependence on voltage difference across the gap junction (V_{GJ}) and time. Alternatively, GJ may be modeled according to model of (Snipas et al., 2020).

### Tight Junctions

Multicellular organs and organisms develop very low-permeability TJ at their exterior boundary, which are involved in creating the important TEP voltage gradient across the organ/organism boundary. In the presently disclosed method, the degree of movement of ions in both chemical and electrical gradients was handled by considering three interconnected, but distinct transport pathways (transmembrane, intercellular, extracellular), with the possibility for spatially varying diffusion coefficients within extracellular regions, with low diffusion at the boundary simulating the presence of TJ.

### Electroosmosis

Electroosmotic flows are a hypothesized transport mechanism in biological systems (Andreev, 2013). The presently disclosed method assumes that electroosmosis may occur through small channel structures of the heterogeneous tissue, such as gap junctions between cells (gap junction radius r_{gj}˜5 to 8 nm) and the narrow channels (d_{ecm}˜10 to 30 nm) formed by extracellular spaces.

A modified version of the Hagen-Poiseuille equation (Gao et al., 2011) was used to estimate electroosmotic fluid flows between the small channels represented by gap junction connected cells or extracellular spaces:

$${\vec{u}}_{o} = \frac{π {r}^{4}}{8 μ} {\vec{F}}_{e} . \qquad Equation 2 l$$

Where a volume force generated by electrostatic forces resulting from a voltage gradient between two cells or extracellular spaces is:

$${\vec{F}}_{e} = {ρ}_{e} \vec{E} . \qquad Equation 22$$

As mass cannot be created or destroyed, fluid flow velocity must be a divergence-free field, which physically corresponds to the development of internal pressures resisting fluid flow. The internal pressure was estimated as:

$${P}_{int} = {∇}^{- 2} ( ∇ \cdot {\vec{u}}_{o} ) . \qquad Equation 23$$

The gradient of the internal pressure was used to correct the velocity calculated from equation (30), yielding the final estimate for electroosmotic fluid velocity:

$$\vec{u} = {\vec{u}}_{o} - ∇ {P}_{int} . \qquad Equation 24$$

Electroosmotic fluid velocities were treated separately in the intra- and extracellular spaces.

### Activating and Inhibiting Effects of Chemical Substances

The concentration of chemical substances within the cells and the environment can influence the activation of ion channels, gap junctions, the growth and decay of another chemical, the rate of a chemical reaction, the rate of flux of a membrane pump, or the rate of flux of a membrane transporter.

The activating influence β^{A} of a substance A upon any of the mechanisms listed above is described by the Hill function with the form:

$${α}^{A} = \frac{{( \frac{[ A ]}{{K}_{A}} )}^{{n}_{A}}}{1 + {( \frac{[ A ]}{{K}_{A}} )}^{{n}_{A}}} . \qquad Equation 25$$

Where [A] is the concentration of substance A, K_{A} is the half activation constant of substance A, n_{A} is the Hill coefficient of substance A.

The inhibiting influence β^{A} of a substance A upon any of the mechanisms listed above is described by the Hill function with the form:

$${β}^{A} = \frac{1}{1 + {( \frac{[ A ]}{{K}_{A}} )}^{{n}_{A}}} . \qquad Equation 26$$

For example, α^{A} could represent the agonistic effect of the chemical acetyl-choline, on the diffusion rate of a sodium ion channel.

### Generation and Decay of Chemical Substances

Cells can synthesize chemical substances. The presence of these substances, and their interaction with other substances can be specified by the user. The change in concentration of a substance [A] which is generated within a cell is described by the differential equation:

$$\frac{d [ A ]}{d t} = \sum {α}^{i} ( {c}_{i} ) {r}_{A}^{i} - \sum {β}^{j} ( {c}_{j} ) {d}_{A}^{j} . \qquad Equation 27$$

Where

$$\sum {α}^{i} ( {c}_{i} ) {r}_{A}^{i}$$

is the sum over i different concentration-dependent growth rates αⁱ(cᵢ) which influence the production of the substance A, cᵢ is the concentration of substance i,

$${r}_{A}^{i}$$

is the maximum growth rate of A given the presence of substance i. The term

$$\sum {β}^{j} ( {c}_{j} ) {d}_{A}^{j}$$

is the sum over j different concentration-dependent decay rates

$${β}^{j} ( {c}_{j} ) {d}_{A}^{j}$$

which influence the decay of the substance A, cⱼ is the concentration of substance j, and

$${d}_{A}^{j}$$

is the maximum decay rate of A from the presence of substance j.

### Chemical Reactions

Chemical reactions comprise the conversion of the concentrations of chemical substances into the concentrations of other chemical substances and are described using rate equations. For example, the reaction between two chemical substances to produce two additional substances is described by the chemical equation:

$$a A + b B ↔ c C + d D . \qquad Equation 28$$

Where lower case letters refer to the stochiometric coefficients.

The change in concentration of the reactants is described by the differential equations:

$$\begin{matrix}\frac{d [ A ]}{d t} = - {k}_{f} [ A ] [ B ] + {k}_{r} [ C ] [ D ] \\ \frac{d [ B ]}{d t} = - {k}_{f} [ A ] [ B ] + {k}_{r} [ C ] [ D ]\end{matrix} . \qquad Equation 29 - 30$$

The change in concentration of the products is described by the differential equations:

$$\begin{matrix}\frac{d [ C ]}{d t} = {k}_{f} [ A ] [ B ] - {k}_{r} [ C ] [ D ] \\ \frac{d [ D ]}{d t} = {k}_{f} [ A ] [ B ] - {k}_{r} [ C ] [ D ]\end{matrix} . \qquad Equation 31 - 32$$

Where k_{f} is the forward rate of the reaction and kᵣ is the reverse rate of the reaction. The forward rate of the reaction is defined in terms of the Hill functions for the reactants:

$${k}_{f} = {k}_{max , f} ( \frac{{( \frac{[ A ]}{{K}_{A}} )}^{{n}_{a}}}{1 + {( \frac{[ A ]}{{K}_{A}} )}^{{n}_{a}}} ) ( \frac{{( \frac{[ B ]}{{K}_{B}} )}^{{n}_{b}}}{1 + {( \frac{[ B ]}{{K}_{B}} )}^{{n}_{b}}} ) . \qquad Equation 33$$

Where k_{max,f} is the maximum forward rate, nₐ and n_{b} are the Hill coefficients of reactants A and B, and K_{A} and K_{B} are the half-saturation constants for reactants A and B.

Similarly, the reverse rate is defined in terms of the Hill functions of the products:

$${k}_{r} = {k}_{max , r} ( \frac{{( \frac{[ C ]}{{K}_{C}} )}^{{n}_{c}}}{1 + {( \frac{[ C ]}{{K}_{C}} )}^{{n}_{c}}} ) ( \frac{{( \frac{[ D ]}{{K}_{D}} )}^{{n}_{d}}}{1 + {( \frac{[ D ]}{{K}_{D}} )}^{{n}_{d}}} ) . \qquad Equation 34$$

Where k_{max,r} is the maximum reverse rate, n_{c} and n_{d} are the Hill coefficients of products C and D, and K_{C} and K_{D} are the half-saturation constants for reactants C and D.

For an irreversible reaction, kᵣ=0.

### Kinetic Implementations of the Present Disclosure

The Nernst-Planck differential equation, describing the rate of change in the concentration of an ion, can also be represented in terms of flux (Φ):

$${Φ}_{NP} = ∇ \cdot ( P ∇ c + \frac{PzF}{RT} E ) . \qquad Equation 35$$

where ∇· is the divergence operator, ∇ is the gradient operator, c is the concentration of an ion or chemical, P is the spatially-varying permeability of an ion or chemical, z is the valence, charge, T is temperature, F is Faradays constant, and R is the universal gas constant, and E is the electric field.

Electrodiffusion can take place between discretized compartments of intra- or extracellular space, where it is described fully by Equation 35. However, electrodiffusion may take place across a boundary separated by a membrane, for example via ion channels or gap junctions, in which case it is more appropriately described using the Goldman-Hodgkin-Katz Flux equation (GHK Flux equation):

$${Φ}_{GHK} = P \frac{zVF}{RT} ( \frac{{c}_{cell} - {c}_{env} exp ( - \frac{zVF}{RT} )}{1 - exp ( - \frac{zVF}{RT} )} ) . \qquad Equation 36$$

where V can be membrane potential Vₘ or gap junction potential Vⱼ.

Ultimately, all fluxes must be translated into changes in concentration within a kinetic scheme. By using kinetic schemes, the equations governing the tissue system can be written using repeated identical components, which greatly increases the speed of solving these equations on a GPU. Accordingly, changes in concentrations of chemicals or ions are preferably described using kinetic equations:

$$\frac{{dc}_{i}}{dt} = - {\sum }_{j} {r}_{ij} {c}_{i} + {\sum }_{j} {r}_{ji} {c}_{i} . \qquad Equation 37$$

where rᵢⱼ is the (possibly voltage or concentration dependent) rate of the reaction between state i and state j that leads to a loss of state i, and rⱼᵢ is the rate of the reverse reaction from state j to i that leads to a gain of state i. This scheme naturally describes chemical reactions that may occur in the cell and in the external environment, however it may further be extended to cover ion or chemical flux, and the kinetic states involved in ion channels, gap junctions, and transporters. Nernst-Planck flux can be translated into a kinetic scheme by noting that the gradient operator ∇ implies that a difference in concentrations (cᵢ−cⱼ) contributes to the concentration change, and the divergence operator being equal to zero (∇·( )=0) implies conservation of a quantity (i.e., if there is an equation stating that one concentration increases, there must be another equation stating that a corresponding concentration decreases). In addition, the changes in concentration must be scaled by the volume of the cell or environment in which the ion/chemical concentration is present (Vol_{ci}) and the surface area of the boundary between regions i and j (Aᵢⱼ) over which a change in concentration occurs. This method can be seen in Equations 38 and 39, which may correspond to the ion concentrations in two compartments of the external environment. Ions can flow between these compartments via diffusion (−Pcᵢ+Pcⱼ) and via the force imparted on charged particles by the electric field

$$( \frac{{Pz}_{i} F}{RT} {E}_{ij}^{′} {c}_{i} + \frac{{Pz}_{j} F}{RT} {E}_{ji}^{′} {c}_{j} ) .$$

However, for this scheme to be physically consistent with Equation 35, the electric field must be modified such that

$${E}_{ij}^{′} = max ( {E}_{ij} , 0 ) ,$$

or in other words, the electric field is always non-negative to avoid double-counting its contribution.

$${Vol}_{ci} \frac{{dc}_{i}}{dt} = ( - {PA}_{ij} {c}_{i} + {PA}_{ji} {c}_{j} ) - \frac{{PA}_{ij} {z}_{i} F}{RT} {E}_{ij}^{′} {c}_{i} + \frac{{PA}_{ji} {z}_{j} F}{RT} {E}_{ji}^{′} {c}_{j} . \qquad Equation 38$$

$${Vol}_{cj} \frac{{dc}_{j}}{dt} = ( - {PA}_{ij} {c}_{j} + {PA}_{ji} {c}_{i} ) - \frac{{PA}_{ij} {z}_{j} F}{RT} {E}_{ji}^{′} {c}_{j} + \frac{{PA}_{ji} {z}_{i} F}{RT} {E}_{ij}^{′} {c}_{i} . \qquad Equation 39$$

Diffusion across a membrane, described by the Goldman-Hodgkin-Katz flux equation, can similarly be described as a kinetic scheme. For example, diffusion between a cell (i) and an adjacent environment (j) is given by:

$${Vol}_{ci} \frac{{dc}_{i}}{dt} = - {PA}_{ij} {Bc}_{i} + {PA}_{j i} {Bc}_{j} exp ( - \frac{z {V}_{m} F}{RT} ) \qquad Equation 40$$

$${Vol}_{cj} \frac{{dc}_{j}}{dt} = - {PA}_{j i} {Bc}_{j} exp ( - \frac{z {V}_{m} F}{RT} ) + {PA}_{ij} {Bc}_{i} .$$

$$B = \frac{\frac{z {V}_{m} F}{RT}}{1 - exp ( - \frac{z {V}_{m} F}{RT} )} . \qquad Equation 41$$

### Net Charge Density, Membrane Potentials, and Electric Fields

For each cell or membrane compartment, the net charge density is calculated by summing the product of each ion or chemical and its valence:

$$ρ = {\sum }_{i} F {z}_{i} {c}_{i} . \qquad Equation 42$$

Cell potentials are calculated by assuming that, because the cell contains an electrolyte solution, the excess charge within a cell resides on the boundary layer close to the surface. In this case, the cell may be treated as a parallel plate capacitor, and the following equation may be used to determine the cell potential:

$${ψ}_{cell} = \frac{{ρ}_{cell} {Vol}_{cell}}{{C}_{m} {A}_{cell}} . \qquad Equation 43$$

where ψ_{cell} is the cell potential, Vol_{cell} is the cell volume, Cₘ is the specific membrane capacitance, and A_{cell} is the cell surface area.

Extracellular potentials are calculated by using a volume conductor model for the discretized extracellular space. The following relationship is used:

$$∇ \cdot ( σ ( r ) ∇ {ψ}_{env} ( r , t ) ) = - {I}_{m} ( r , t ) . \qquad Equation 44$$

where σ(r) is the conductivity of the extracellular medium which may vary with position r, Iₘ(r,t) are the membrane currents at each point in space, and ψₑₙᵥ is the extracellular potential which is solved for implicitly.

Membrane potentials are then the difference between intracellular and extracellular potentials:

$${V}_{m} = {ψ}_{cell} - {ψ}_{env} . \qquad Equation 45$$

Gap junction potentials are defined the difference between the potentials of adjacent cells:

$${V}_{J} = {ψ}_{cell , 0} - {ψ}_{cell , 1} . \qquad Equation 46$$

### Electrogenic Mechanisms

In the simulator, ionic currents between compartments (cells or environment) are produced by electrogenic mechanisms which are modelled using linear kinetic schemes. Electrogenic mechanisms can include ion channels and gap junctions, where the kinetic scheme links voltage, or the concentration of some ligand, to the conductance of the channel. Ion fluxes are then produced via diffusion along the electrochemical gradient. Electrogenic mechanisms can also include transporters, in which ions bind to substrates which then undergo conformational changes to move ions across the membrane. In the case of primary active transport (pumps), ion flux is produced by the chemical energy extracted from adenosine triphosphate. In the case of secondary active transport, energy is extracted from the concentration gradient of one set of ions and used to induce the flux of another set of ions.

### Kinetic Schemes

Kinetic schemes are a network-based approach to describing the transitions between discrete states of a system. Their versatility allows them to be used to describe a vast range of physical processes. Let P=[p₁, p₂, . . . . PN] denote the vector of occupancies (mole fraction or probability) of N discrete states. Transitions between these states occur with a first order rate coefficient kᵢⱼ yielding the master equation:

$$\frac{dP}{dt} = QP . \qquad Equation 47$$

where Q is the transition matrix with off diagonal entries kᵢⱼ indicating transition probabilities from one state to another, and diagonal entries kᵢᵢ being the negative column sum of the off diagonal entries. This is the matrix form of Equation 37. Rate coefficients can further be exponential functions of voltage, temperature, or ligand concentration:

$${k}_{ij} = {k}_{ij 0} {\prod }_{k} {c}_{k} exp ( α ( {V}_{m} , pH , T ) ) . \qquad Equation 48$$

where α is a linear function, and Πₖ cₖ denotes the product of the k ligands which affect the rate kᵢⱼ. Πₖ cₖ maybe replaced by 1 when no ligands are involved. The dependence of a mechanism on pH can be introduced by having transition rates which depend on hydrogen concentration.

For example, the following kinetic scheme, represented as a directed graph, may be written as a transition matrix (Equation 49):

For example, the following kinetic scheme, represented as a directed graph, may be written as a transition matrix (Equation 49):

$$Q = ( \begin{matrix}\frac{- a - d}{{v}_{0}} & f & c \\ a & \frac{- f - b}{{v}_{1}} & e \\ d & b & \frac{- c - e}{{v}_{2}}\end{matrix} ) . \qquad Equation 50$$

where v₀,v₁,v₂ are the volumes of states 0, 1, and 2 respectively. For most kinetic schemes, states are interpreted as probabilities and so vᵢ=1. However, when a state represents a concentration, vᵢ will represent the volume of the compartment to which that concentration applies.

### Ion Channels

Ion channels are modelled as dynamic diffusion rates located in cell membranes facilitating electro-diffusion between the cell and environment. The diffusion rate of an ion channel can change as a function of voltage or ligand concentration via a kinetic scheme. A diffusion rate r of cell membrane can be related to a state kᵢⱼ∈[0,1] of a kinetic scheme by r=D kᵢⱼ where D is the maximum ionic diffusion rate across the membrane.

### Gap Junctions

Similar to ion channels gap junctions are modelled as dynamic diffusion rates between two connected cells. A diffusion rate g of cell membrane can be related to a state kᵢⱼ∈[0,1] of a kinetic scheme by g=Gkᵢⱼ where G is the maximum ionic diffusion rate across the gap junction.

### Transport

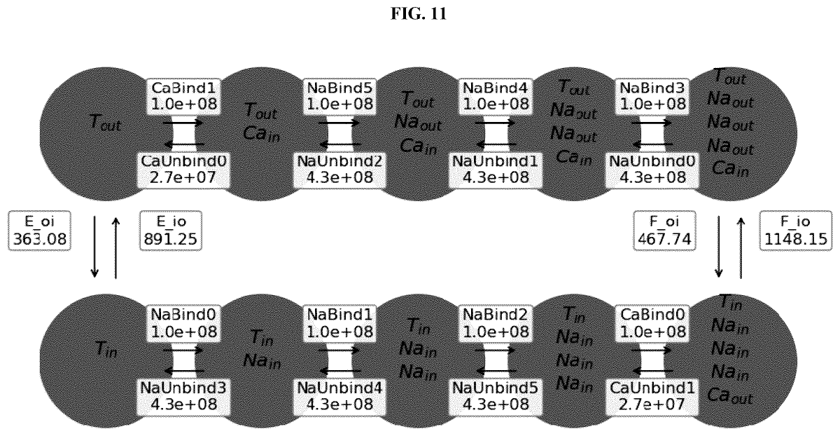

The transport of ions or chemical across the cell membrane is described by kinetic schemes wherein ions bind to a protein complex on the cell membrane. Transport occurs as an ion or chemical binds on one side of the membrane, is translocated to the opposite side, and then dissociates. This process may occur in multiple steps with multiple binding, unbinding, and translocation operations, as seen in FIG. 11.

### Chemical Reactions

Ions or chemicals may react with one another, within the cell or external environment and in doing so lead to a change in the concentration of ions or chemicals. These types of reactions can range from the simple the dissociation of carbon dioxide in water into carbonate and hydrogen ions, or the Krebs cycle.

### Network Properties and Structure

A “network” is an in-silico representation comprising nodes (cells and environment volumes) and edges (couplings) specified by a schema that separates node and edge characteristics. Each node is parameterized by cell geometry (size, shape, surface area), cell type/identity, a repertoire of molecular bioelectric components (including channel or transporter types, densities, conductances and transport rates). From these specifications, higher-order node dynamics may be defined, including excitability, bistability, and oscillatory regimes. Each edge specifies which nodes are connected, the coupling modality (e.g., gap junctions, ion channels, transporters, pumps), and coupling strength (e.g., junctional conductance, ion channel conductance, transporter rate). The network may instantiate actual biological tissue, an abstract tissue model for theoretical exploration, or a testbed for evaluating interventions. Network analysis may be performed to compute connectivity metrics, including degree distributions and related measures useful for characterizing topology and predicting function.

The presently disclosed methods may further characterize and exploit emergent behaviours arising from specified node and edge parameters, including coherent network-level oscillations, propagation of bioelectric wavefronts, interface dynamics, and bistability that can arise between coupled cells with complementary ion-channel compositions (e.g., a node containing a sodium channel coupled to a node containing a potassium channel via a gap junction behaving as a single bistable unit through ionic diffusion). In some embodiments the network is configured to perform computations, wherein ordered stimulation of selected nodes produces predefined spatiotemporal patterns or logic-gate-like operations. Manipulations may include altering ion-channel composition or conductance at one or more nodes (including addition, removal, blockade, or activation), modifying coupling modality or strength on selected edges, rewiring connectivity by changing which nodes are connected, applying agents to modulate channel activity in targeted cells or groups of cells, and delivering electrical stimulation to selected nodes or groups. Parameter sweeps and closed-loop adjustments may be used to elicit, suppress, or stabilize specified emergent behaviours.

### Automatic Differentiation

Automatic differentiation (AD) performs a non-standard interpretation of a given computer program by replacing the domain of the variables to incorporate derivative values and redefining the semantics of the operators to propagate derivatives per the chain rule of differential calculus. All numerical computations are ultimately compositions of a finite set of elementary operations for which derivatives are known (Verma, 2000; Griewank and Walther, 2008), and combining the derivatives of the constituent operations through the chain rule gives the derivative of the overall composition. Computational graphs, which track the output and order of the mathematical operations, for the basis of the AD technique.

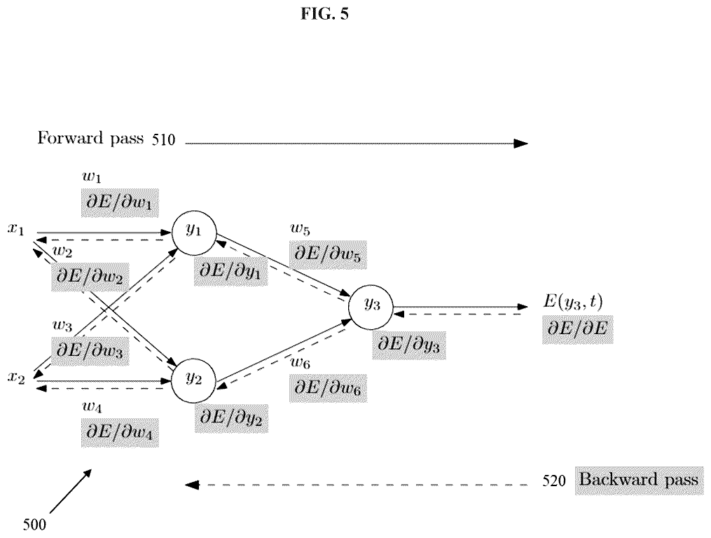

AD in the reverse accumulation mode corresponds to a generalized backpropagation algorithm, in that it propagates derivatives backward from a given output. This is done by complementing each intermediate (working) variable vᵢ with an adjoint vᵢ=∂yⱼ/∂vᵢ, which represents the sensitivity of a considered output vᵢ with respect to changes in vᵢ. In the case of backpropagation, y would be a scalar corresponding to the error E (FIG. 5).

In reverse mode AD, derivatives are computed in the second phase of a two-phase process. In the first phase, the original function code is run forward, populating intermediate variables vᵢ and recording the dependencies in the computational graph through a book-keeping procedure. In the second phase, derivatives are calculated by propagating adjoints vᵢ in reverse, from the outputs to the inputs.

AD reverse mode is significantly less costly to evaluate (in terms of operation count) than the forward mode for functions with a large number of inputs. In the extreme case of f: [symbol] ⁿ→ [symbol] , only one application of the reverse mode is sufficient to compute the full gradient ∇f=(∂y/∂x₁, . . . , ∂y/∂xₙ), compared with the n passes of the forward mode needed for populating the same. Because machine learning practice principally involves the gradient of a scalar-valued objective with respect to a large number of parameters, this establishes the reverse mode, as opposed to the forward mode, as the mainstay technique in the form of the backpropagation algorithm. Additional information can be found in Reference 2, which is hereby incorporated by reference in its entirety.

### Modes for Carrying Out the Disclosure

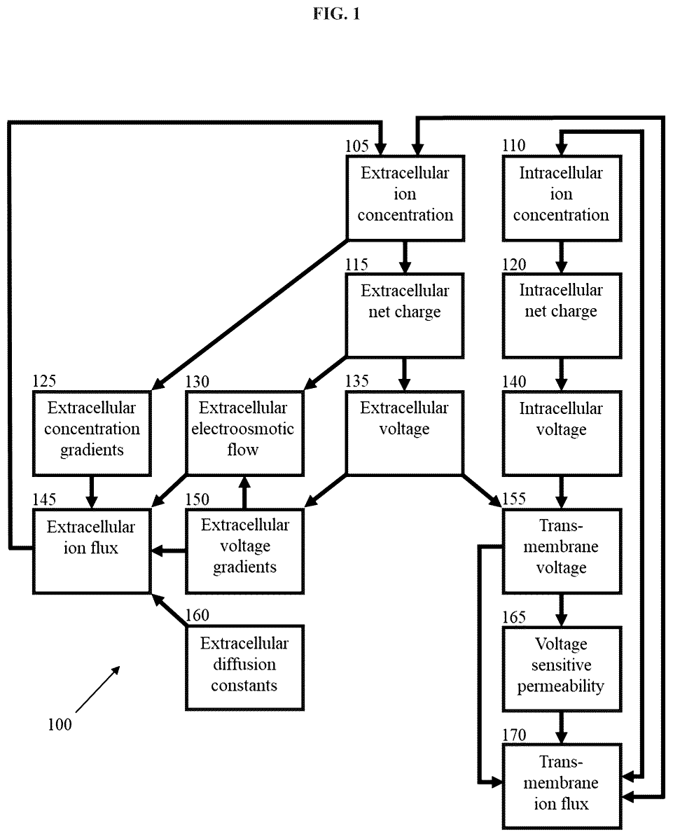

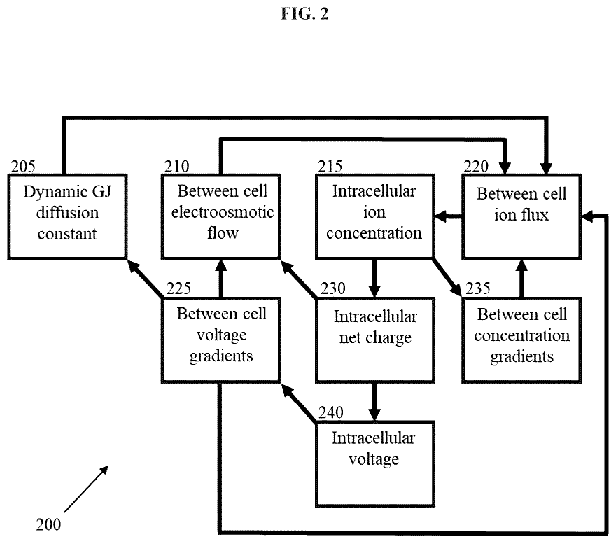

FIGS. 1-2 illustrate an example block diagram of computation steps of the present disclosure, in accordance with some embodiments. Computation steps 100 and 200 may be implemented by computer environment 400.

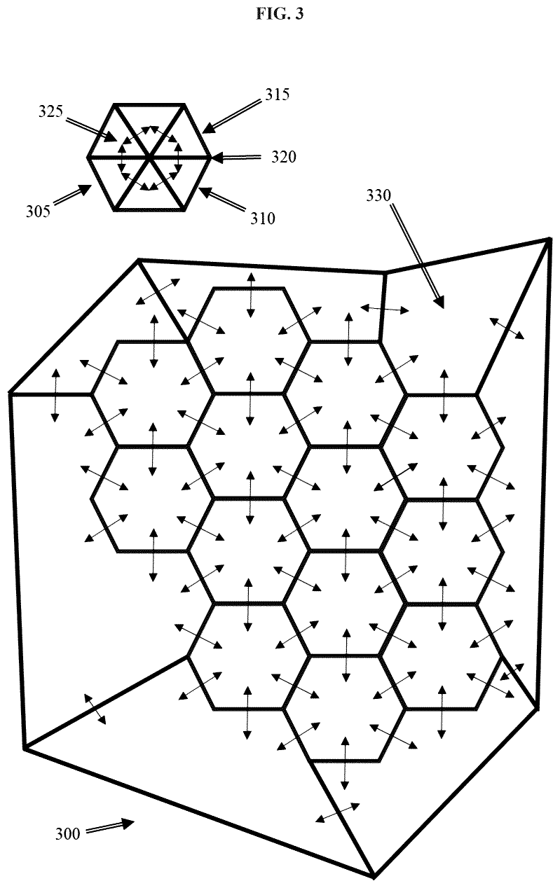

FIG. 3 illustrates one embodiment of an array of cells 300, each cell 305 comprising a volume, intracellular ion concentration, membrane characteristics, and shared small extracellular spaces 315 and 320 exterior to facets of cell 305's perimeter. In some embodiments, the electrodiffusion of ions between small extracellular spaces 310 and 315 at 320 is calculated using the Nernst Planck equation. In some embodiments, cell 305 is divided into sub compartments 325, enabling polarization of cell 300 through Nernst Planck equations calculated (e.g., by the arrows) between sub compartments.

Arrows between cells in array of cells 300 denote that neighboring cells may receive and transmit electrophysiological information to other cells, either directly via GJ or indirectly via the shared small extracellular space. Arrows between cells and large extracellular spaces 330 on the border of the array of cells 300 denote that cells may receive and transmit electrophysiological information with large extracellular spaces through the shared small extracellular space. Arrows between large extracellular space 330 and neighboring large extracellular spaces denote that large extracellular spaces may receive and transmit electrophysiological information with large extracellular spaces directly. In some embodiments, electrodiffusion of ions between large extracellular spaces is calculated using the Nernst Planck equation.

In some embodiments, the user inputs specific extracellular ion concentrations 105 and intracellular ion concentrations 110 for each of the cells 305 and associated extracellular spaces of the array of cells 300. In some embodiments, extracellular net charge is calculated at step 115 and intracellular net charge is calculated at step 120 by Faraday sum equations using extracellular ion concentrations 105 and intracellular ion concentrations 110. In some embodiments, extracellular voltage is calculated at step 135 and intracellular voltage is calculated at step 140 using extracellular net charge 115 and intracellular net charge 120. In some embodiments, extracellular voltage gradients are calculated at step 150 by gradient operator equations using extracellular voltage 135. In some embodiments, extracellular electroosmotic flow is calculated at step 130 by multiplying extracellular voltage gradient 150 with extracellular net charge 115 and by using Stokes flow equations. In some embodiments, extracellular concentration gradients are calculated at step 125 by gradient operator equations using extracellular ion concentration 105. In some embodiments, extracellular spatially variable diffusion constants are applied at step 160. In some embodiments, extracellular ion flux is calculated at step 145 by Nernst Planck flux equations using extracellular concentration gradients 125, extracellular electroosmotic flow 130, extracellular voltage gradients 150, and extracellular diffusion constants 160. In some embodiments, transmembrane voltage is calculated at step 155 by subtracting extracellular voltage 135 from intracellular voltage 140. In some embodiments, voltage sensitive dynamic membrane permeability is calculated at step 165 by using membrane gating equations. In some embodiments, transmembrane ion flux is calculated at step 170 by GHK flux equations and ion pump equations using transmembrane voltage 155, voltage sensitive permeability 165, extracellular ion concentration 105, and intracellular ion concentration 110.

In some embodiments, extracellular ion concentration 105 is calculated and updated at timestep t by divergence operator equations using extracellular ion flux 145 and transmembrane ion flux 170. In some embodiments, intracellular ion concentration 110 is calculated and updated at timestep t by divergence operator equations using transmembrane ion flux 170.

In some embodiments, between cell concentration gradients are calculated at step 235 by gradient operator equations using intracellular ion concentration 215. In some embodiments, between cell voltage gradients are calculated at step 225 by gradient operator equations using intracellular voltage 240. In some embodiments, between cell electroosmotic flow is calculated at step 210 by multiplying intracellular net charge 230 by between cell voltage gradients 225 and by using Stokes flow equations. In some embodiments, a voltage sensitive dynamic gap junction diffusion constant is calculated at step 205 by applying gap junction gating functions to between cell voltage gradient 225. In some embodiments, between cell ion flux is calculated at step 220 by Nernst Planck flux equations using dynamic GJ diffusion constant 205, between cell electroosmotic flow 210, between cell voltage gradients 225, and between cell concentration gradients 235.

In some embodiments, intracellular ion concentration 215 is calculated and updated at timestep t by divergence operator equations using between cell ion flux 220.

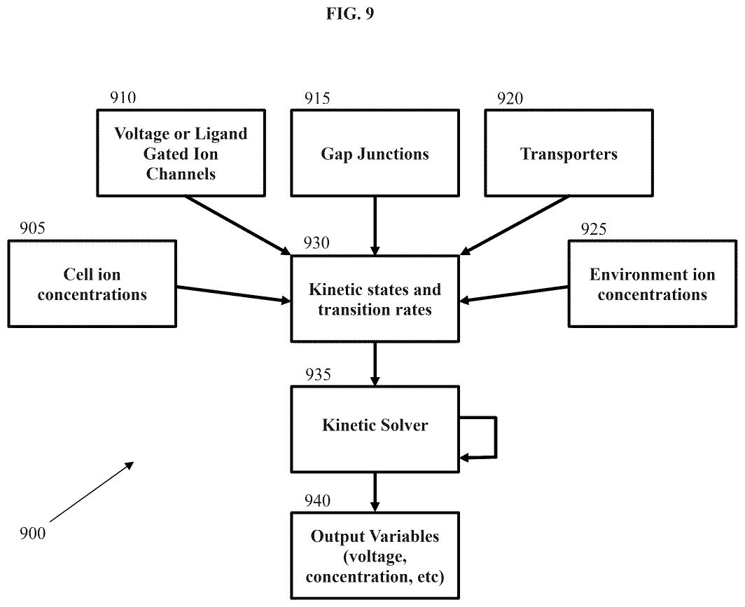

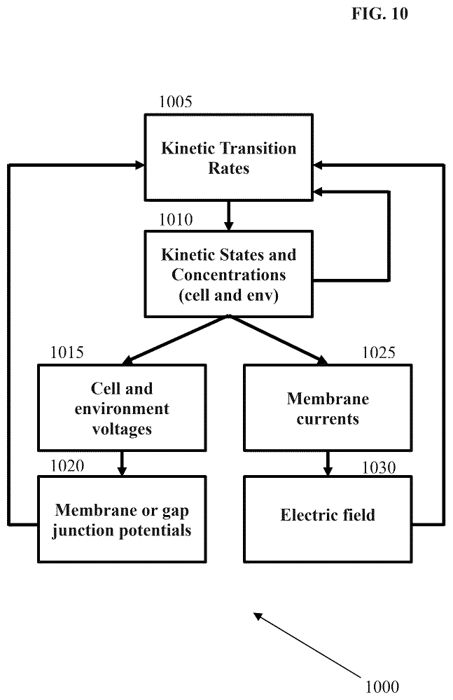

FIGS. 9-10 illustrate an example block diagram of alternative and preferred computation steps of the present disclosure. Computation steps 900 and 1000 may also be implemented by computer environment 400.

In some embodiments, specific extracellular chemical substance and/or ion concentrations 925 and intracellular chemical substance and/or ion concentrations 905 are assigned by a processor to each cell 305 and the associated extracellular spaces of the array of cells 300. The processor may also assign values for permeabilities associated with ion channels 910, gap junctions 915, and/or transporters 920. A kinetic state and/or transition rate for each of the membrane chemical substance and/or ionic permeabilities may be assigned and/or calculated by the processor at step 930, for input into kinetic solver 935, which iteratively calculates and updates the kinetic states and/or transition rates to output a cell membrane voltage and/or ion concentration at step 945.

In some embodiments, a kinetic state 1010 and transition rate 1005 is iteratively calculated and updated by the processor, based on a change in chemical substance and/or ion concentration 1010 over time, t, and based on a change in membrane potential 1020 and electric field 1030 over time, t. In some embodiments, the change is membrane potential 1020 is calculated from cell and environmental voltages 1015, which in turn is calculated from kinetics states and chemical substance and/or ion concentrations 1010. In some embodiments, the change in electric field 1030 is calculated from membrane currents 1025, which in turn is calculated from kinetics states and chemical substance and/or ion concentrations 1010.

In any of the aforementioned embodiments, the membrane permeability may correspond to the permeability of a voltage or ligand gated ion channel, a gap junction, and/or and ion or chemical substance transporter; the kinetic transition rate may be voltage or concentration dependent; and the kinetic state and transition rate for each of the membrane ionic and chemical substance permeabilities may be updated simultaneously.

Various actions discussed herein, such as methods and processes discussed herein may be performed (at least partially) by one or more processors implementing/utilizing one or more artificial intelligence models.

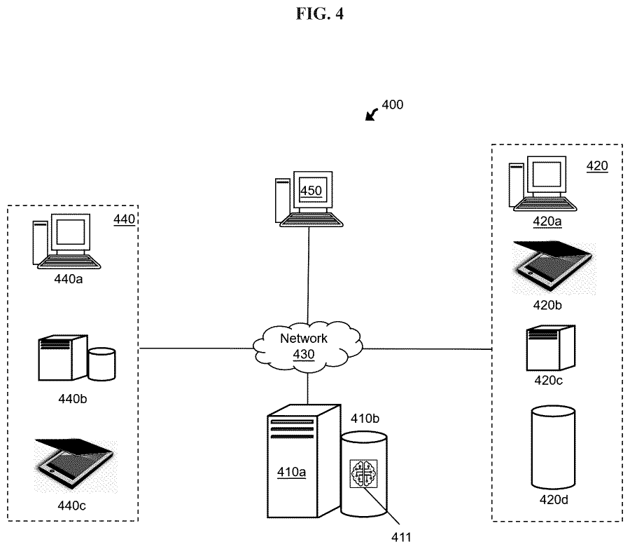

FIG. 4 illustrates an example computer environment 400 that can be used to provide a network-based implementation of the methods and processes described herein. Specifically, FIG. 4 illustrates components of a system 400 for a bioelectric response simulation system 400 (system 400), according to an embodiment. The system 400 may include an analytics server 410a, system database 410b, a machine learning model 411, electronic data sources 120a-d (collectively electronic data sources 420), end-user devices 440a-c (collectively end-user devices 440), and an administrator computing device 450.

The system 400 is not confined to the components described herein and may include additional or other components not shown for brevity, which are to be considered within the scope of the embodiments described herein.

The above-mentioned components may be connected to each other through a network 130. Examples of the network 430 may include, but are not limited to, private or public local-area-networks (LAN), wireless LAN (WLAN) networks, metropolitan area networks (MAN), wide-area networks (WAN), and the Internet. The network 430 may include wired and/or wireless communications according to one or more standards and/or via one or more transport media. Communication over the network 430 may be performed in accordance with various communication protocols such as Transmission Control Protocol and Internet Protocol (TCP/IP), User Datagram Protocol (UDP), and Institute of Electrical and Electronics Engineers (IEEE) communication protocols. In one example, the network 430 may include wireless communications according to Bluetooth specification sets or another standard or proprietary wireless communication protocol. In another example, the network 430 may also include communications over a cellular network, including, e.g., GSM (Global System for Mobile Communications), CDMA (Code Division Multiple Access), or EDGE (Enhanced Data for Global Evolution) networks.

The analytics server 410a may generate and display an electronic platform configured to receive information and output results of execution of the machine learning model 411. The electronic platform may include a graphical user interface (GUI) displayed on the electronic data sources 420, the end-user devices 440, and/or the administrator computing device 450. An example of the electronic platform generated and hosted by the analytics server 410a may be a web-based application or a website configured to be displayed on various electronic devices, such as mobile devices, tablets, personal computers, and the like. Simply put, the analytics server 410a may implement the platform to receive data and instructions from end users (using the end-user devices 440). The analytics server 410a may then execute the machine learning model 411 accordingly and display the results (e.g., a list of “suitable” or “best” mutations for tracking a patient's progress over the course of treatment or cancer recurrence) on the platform.

The analytics server 410a may be any computing device comprising a processor and non-transitory, machine-readable storage capable of executing the various tasks and processes described herein. The analytics server 410a may employ various processors such as a central processing unit (CPU) and graphics processing unit (GPU), among others. Non-limiting examples of such computing devices may include workstation computers, laptop computers, server computers, and the like. While the system 400 includes a single analytics server 410a, the analytics server 410a may include any number of computing devices operating in a distributed computing environment, such as a cloud environment.

The electronic data sources 420 may represent various sources that contain, retrieve, and/or access data needed to train the machine learning model 411. For instance, the analytics server 410a may use a laboratory computer 420a, medical professional device 420b, server 420c (associated with a laboratory), and/or database 420d (associated with a research lab, a clinic, and/or any third party providing data) to retrieve and receive data. As used herein, the electronic data sources may include any electronic source containing data (e.g., WGS data) that can be used to generate a training dataset in order to (ultimately) train the machine learning model 411. Even though referred to herein as “laboratory” devices, these devices may not always be operated in laboratories. Therefore, no limitation is intended by this term.

When generating the training data, the analytics server 410a may execute various algorithms to translate raw data received or retrieved from the electronic data sources 420 into machine-readable objects that can be stored and processed by other analytical processes as described herein.

End-user devices 440 may be any computing device comprising a processor and a non-transitory, machine-readable storage medium capable of performing the various tasks and processes described herein. Non-limiting examples of an end-user device 440 may be a workstation computer, laptop computer, tablet computer, or server computer. During operation, various users may use end-user devices 440 to access the GUI operationally managed by the analytics server 410a. Specifically, the end-user devices 440 may include laboratory computer 140a, laboratory server 440b, and a user device 440c. Even though referred to herein as “end-user” or “laboratory” devices, these devices may not always be operated by end-users or in laboratories. Therefore, no limitation is intended by these terms.

The administrator computing device 450 may represent a computing device operated by a system administrator. The administrator computing device 450 may be configured to display various attributes and predictions generated by the analytics server 410a (e.g., various analytic metrics determined during training of one or more machine learning models and/or systems); monitor various models 411 utilized by the analytics server 410a, electronic data sources 120, and/or end-user devices 440; review feedback; and/or facilitate training or retraining (calibration) of the machine learning model 411 that are maintained by the analytics server 410a.

The machine learning model 411 may be stored in the system database 410b. The machine learning model 411 may be trained using data received or retrieved from the electronic data sources 420 and may be executed using data received from the end-user devices 440. In some embodiments, the machine learning model 411 may reside within a data repository that is local or specific to a laboratory or an end user (e.g., client). In other embodiments, the machine learning model may be stored centrally where access to its predictions are controlled per client basis.

It should be understood that any alternative and/or additional machine learning model(s) may be used to implement similar learning engines. As described herein, the analytics server 410a may store the machine learning model 411 (e.g., neural networks, random forest, support vector machines, regression models, recurrent models, etc.) in an accessible data repository. The analytics server 410a may retrieve the machine learning model 411 and train it to predict a suitable subset of mutations for tracking a patient's progress over the course of treatment or cancer recurrence. Various machine learning techniques can be used to train the machine learning model 411, such as supervised learning techniques, unsupervised learning techniques, or semi-supervised learning techniques, among others.

The methods and systems discussed herein can be utilized to develop a software tool designed to simulate networks of cells with a particular emphasis on bio-electrics, potentially for investigating processes related to cellular regeneration. The software tool may be utilized to address existing limitations by offering faster simulations and improved compatibility with machine learning methods for analysis. The methods and systems discussed herein provide a unique approach to modeling cellular environments, such as modeling cellular structures and extracellular spaces with distinct properties such as ion concentrations and voltages, suggesting a nuanced and comprehensive simulation framework.

Various steps discussed herein can be augmented using machine learning techniques. For instance, machine learning models can be used for setting up parameters to analyzing results and potential applications such as drug interventions to observe their effects on cell properties.

In operation, the analytics server 410a can be used to collect data associated with various steps discussed with respect to FIGS. 1-2. Once the data is collected, the machine learning model 411 can be trained using supervised or semi-supervised methods.

The training of the machine learning model 411 may involve generating the training dataset on the fly during the simulation processes discussed herein. For instance, the analytics server 410a may execute the simulation process using different input parameters, such as cell structures and drug interventions, and may observe the corresponding simulation outcomes. These outcomes can then be labeled for training the machine learning model 411. The analytics server 410a may then iteratively execute the simulations in order to accumulate a diverse training dataset representing various scenarios and their corresponding outcomes.

This dataset is then used to train the machine learning model to predict simulation outcomes based on input parameters, effectively learning the underlying relationships between different variables in the cellular environment. This iterative process allows the machine learning component to adapt and improve its predictive capabilities over time, enabling more accurate simulations and deeper insights into cellular behavior.

FIG. 5 illustrates an overview of backpropagation 500 in automatic differentiation. Training inputs xᵢ are fed forward, generating corresponding activations yᵢ. An error E 510 between the actual output y₃ and the target output is computed. The error adjoint is propagated backward 520, giving the gradient with respect to the weights ∇_{wi}E=(∂E/∂w₁, . . . , ∂E/∂w₆), which is subsequently used in a gradient-descent procedure. The gradient with respect to inputs ∇ₓᵢE can be also computed in the same backward pass. Further, FIG. 5 illustrates the application of artificial neural network principles to cellular simulation. In our implementation, the gradient descent calculation is used to optimize the weights, which correspond to the variable parameters of cellular components in our simulation. These weights represent the properties of ion channels, gap junctions, ion pumps, and other cellular elements that are modeled and simulated in our system.

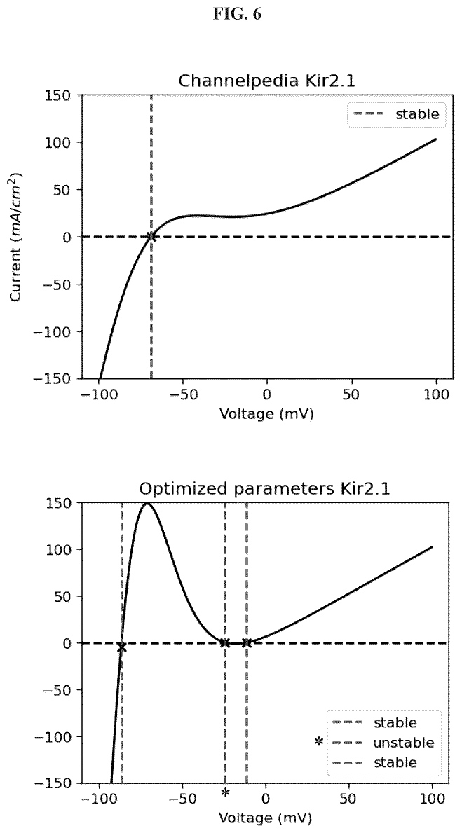

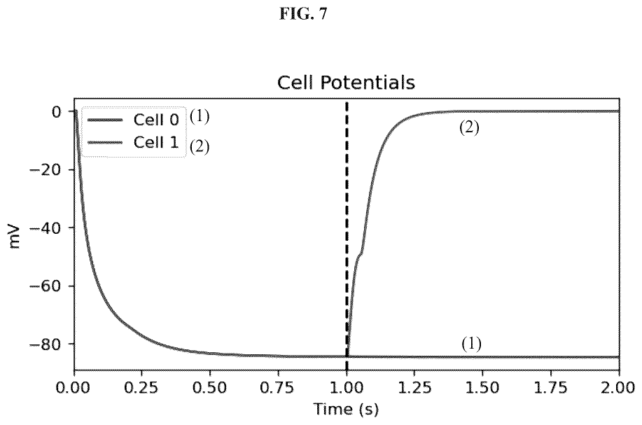

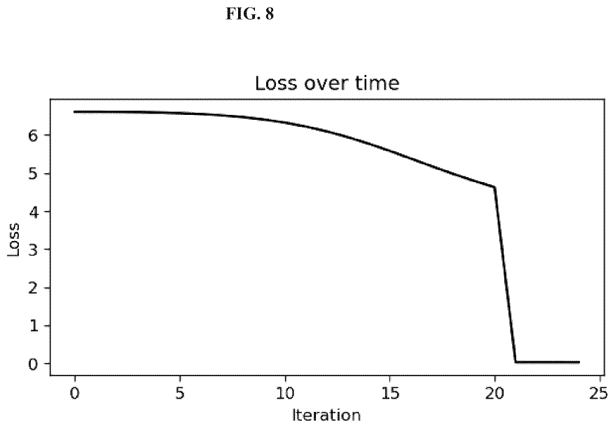

FIGS. 6-8 illustrate a use of the methods and simulator as disclosed herein to identify ion channel parameters that result in an ion channel with a new bistable characteristic. Bistability refers to a characteristic of a system whereby it is capable of possessing two distinct stable states. Applicant sought to determine parameters for the ion channel Kir2.1 which would result in bistability of a modeled cell. The are two traditional approaches to the problem of identifying parameters. The first is to come up with an accurate mathematical model of the system and solve it analytically. This requires a rigorous knowledge of mathematics and may not always be possible for a given biological system. The second approach is to manually search through all possible parameters for the model by brute force. Surprisingly, Applicant efficiently solved the problem with the methods and system disclosed herein. The presently disclosed method and simulator may also be used to identify parameters for ion channels other than Kir2.1.

Applicant set up an experiment with two unconnected cells with the same initial conditions that resulted in a stable depolarized state. After 1 second of simulation, the first cell was given a depolarizing current stimulus lasting 0.4 seconds to switch the cell from a hyperpolarized to a depolarized state. In some embodiments, the depolarization is induced by a current injection, in other embodiments the depolarization is induced by a drug. The simulation was run for a total of 5 seconds, and the value of the cell potential on the final iteration was recorded. Target values desired for the end of the simulation, 0 mV in the stimulated cell and −90 mV in the unstimulated cell, were specified. The voltage values in each cell at the end of the simulation were compared to their target values to produce an error signal. This error signal was back-propagated through the computational graph of the simulation, producing the gradient of the output variables with respect to the inputs. In this case, the inputs were parameter values for the Kir2.1 channel, namely the half-activation voltage and slope, and the half in-activation voltage and slope. In other embodiments, the input parameters comprise a gating exponent, minimum time constant, and/or maximum time constant. These values were initialized to the values acquired from Channelpedia. The parameters were then updated iteratively using the gradient descent algorithm until a bistable voltage state was achieved, which took 21 iterations (the exact number depends on the parameters chosen for the gradient descent algorithm). An Ion channel closely corresponding to the identified parameters may then be identified from a database, such as Channelpedia. Expression of said closely corresponding ion channel in cells or tissues by techniques well known in the art (e.g., gene therapy) therefore provides for living cells having a new bistable characteristic.

Datasets derived from living tissues may also be used to drive computational modeling of electrophysiological behavior across biological systems (in silico analysis). The purpose of these bioelectric simulations is to predict molecular bioelectric targets that are likely to influence, restore, or redirect bioelectric signaling, thereby enabling electroceutical interventions. In this framework, the bioelectric tissue simulator performs an iterative model fitting against a defined bioelectric state, which may include spatial distributions (e.g., voltage gradients) and/or temporal dynamics (e.g., oscillatory or transient signals). A target bioelectric state represents a desired physiological or therapeutic condition. Through successive rounds of model fitting and optimization, the bioelectric tissue simulator identifies and prioritizes molecular bioelectric components that are functionally required to generate and maintain the specified bioelectric profile within the biological model system. These molecular components are then ranked based on their inferred contribution or likelihood to induce the desired bioelectric state, thus creating a prioritized list of potential molecular targets. Such targets are actionable via pharmacological or genetic modulation. In the final step of the workflow, candidate drugs, compounds, or interventions that modulate these prioritized molecular bioelectric components are proposed as electroceuticals (therapeutic agents designed to manipulate endogenous bioelectric signaling). The purpose of this process is the rational prediction of precise molecular targets whose modulation can transform an existing bioelectric state into a known, desirable future state, thereby enabling targeted bioelectric control of tissue function or disease outcomes.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1. Pietak A, Levin M. Exploring Instructive Physiological Signaling with the Bioelectric Tissue Simulation Engine. Frontiers in Bioengineering and Biotechnology. 2016 Jul. 6; 4:55. doi: 10.3389.
2. Baydin A, Pearlmutter B, Radul A, Siskind J. Automatic Differentiation in Machine Learning: a Survey. The Journal of Machine Learning Research. 2018 Apr. 18; 1:5595. doi: 10.5555.

1. A method comprising:
generating a computer model comprising an array corresponding to a set of cells, each cell within the set of cells comprising an intracellular ion concentration and a membrane ionic permeability, the computer model further comprising an extracellular environment interconnecting the array of cells, the extracellular environment comprising an extracellular ion concentration;
assigning a value to each of the intracellular ion concentration, the membrane ionic permeability, and the extracellular ion concentration;
iteratively calculating, by a processor, an intracellular voltage value for at least one cell within the set of cells, wherein, with each iteration, the processor:
calculates a membrane voltage;
calculates a voltage sensitive membrane permeability;
calculates an extracellular ion concentration gradient and a voltage gradient;
calculates a membrane ion flux and an extracellular ion flux over time, t; and
updates the intracellular ion concentration and the extracellular ion concentration of each of the cells based on the calculated membrane ion flux and extracellular ion flux; and
outputting by the processor the intracellular voltage of at least one cell.

2. A system comprising:
a computer-readable medium having instructions that when executed cause a processor to generate a differentiable bioelectric tissue simulator for deriving one or more drug, electric field, or gene therapy interventions that produces a predetermined bioelectric state of a tissue, the differentiable bioelectric tissue simulator executing steps comprising:
generating, by a processor, a computer model comprising an array corresponding to a set of cells, each cell within the set of cells comprising an intracellular ion concentration, an intracellular chemical substance concentration, a membrane ionic permeability, and a membrane chemical substance permeability, the computer model further comprising an extracellular environment interconnecting the array of cells, the extracellular environment comprising an extracellular ion concentration and an extracellular chemical substance concentration;
assigning, by the processor, a kinetic state and transition rate to each of the membrane ionic and chemical substance permeabilities;
iteratively calculating, by the processor, a change in ion and chemical substance concentration over time, t, wherein, with each iteration, the processor:
calculates a membrane potential;
calculates an electric field;
updates, by the processor, the kinetic transition rate and state by the calculated ion and chemical substance concentration, membrane potential, and electric field; and
outputs, by the processor, an intracellular voltage, ion concentration, and/or chemical substance concentration for at least one cell.

3. The system of claim 2, wherein the membrane permeability corresponds to the permeability of a voltage or ligand gated ion channel, a gap junction, and/or and ion or chemical substance transporter; and wherein the kinetic transition rate is voltage or concentration dependent.

4. The system of claim 2, wherein the kinetic state and transition rate for each of the membrane ionic and chemical substance permeabilities is updated simultaneously.

5. The system of claim 2, further comprising: a computational graph comprising one or more mathematical operations; a derivative calculated for each of said one or more mathematical operations, which derivative is calculated by an auto-differentiation process; a partial derivative calculated for each of said one or more mathematical operations, which partial derivative is calculated by a chain rule; one or more variables of the mathematical operations selected by the user; and a gradient descent of the partial derivatives to identify required changes in the selected variables to produce the predetermined bioelectric state.

6. The system of claim 5, wherein the one or more variables correspond to ion channel parameters, and wherein the predetermined bioelectric state corresponds to a bistable membrane voltage.

7. The system of claim 5, wherein the predetermined bioelectric state corresponds to the ionic permeability, chemical substance permeability, intracellular ion concentration, extracellular ion concentration, extracellular chemical substance concentration, intracellular chemical substance concentration, or combinations thereof of cells of a tissue undergoing a biological process, wherein ionic permeability, chemical substance permeability, intracellular ion concentration, ion concentration, extracellular chemical substance concentration, intracellular chemical substance concentration or combinations thereof of cells of the tissue undergoing the biological process is determined by imaging, electrophysiological recordings, measurements of gene expression, measurements of mRNA expression, measurements of protein expression, or combinations thereof, or wherein the predetermined bioelectric state corresponds to the ionic permeability, chemical substance permeability, intracellular ion concentration, extracellular ion concentration, extracellular chemical substance concentration, intracellular chemical substance concentration, or combinations thereof of cells of a tissue resulting from treatment with one or more drug, electric field, or gene therapy interventions, wherein ionic permeability, chemical substance permeability, intracellular ion concentration, extracellular ion concentration, extracellular chemical substance concentration, intracellular chemical substance concentration, or combinations thereof of cells of the tissue undergoing the treatment is determined by imaging, electrophysiological recordings, measurements of gene expression, measurements of mRNA expression, measurements of protein expression, or combinations thereof.

8. The system of claim 7, wherein the one or more drug, electric field, or gene therapy interventions used to determine the predetermined state is different than the one or more drug, electric field, or gene therapy interventions used to derive the predetermined state.

9. The system of claim 7, wherein the biological process is tissue regeneration or a restored natural voltage state.

10. The system of claim 7, wherein the biological process is wound healing, tissue and organ engineering, growth of artificial meat, or an alternative immune system response, and wherein the tissue is selected from the group consisting essentially of an internal organ, a digit, a limb, a muscle, skin, nose, eyes, ears, or brain.

11. A method of treating a wound, injury, or congenital disorder in a patient in need thereof, the method comprising contacting the patient with a pharmaceutical composition, electrical field, or gene therapy, wherein the selection and/or placement of the pharmaceutical composition, electrical field, or gene therapy is determined by the system of claim 2.

12. A method of regenerating a tissue or engineering an organ, the method comprising contacting a group of cells with a pharmaceutical composition, electrical field, or gene therapy, wherein the selection and/or placement of the pharmaceutical composition, electrical field, or gene therapy is determined by the system of claim 2.

13. The method of claim 11, wherein the gene therapy comprises expression of an ion channel corresponding to the ion channel parameters identified by the system of claim 2.

14. The method of claim 13, wherein a bistable membrane voltage is switched from a depolarized to a hyperpolarized membrane voltage, or from a hyperpolarized to a depolarized membrane voltage.

15. A method comprising:
generating a computer model comprising an array corresponding to a set of cells, each cell within the set of cells comprising an intracellular ion concentration and a membrane ionic permeability, the computer model further comprising an extracellular environment interconnecting the array of cells, the extracellular environment comprising an extracellular ion concentration;
assigning a kinetic state and transition rate to the membrane ionic permeability;
iteratively calculating, by a processor, a change in ion concentration over time, t, wherein, with each iteration, the processor:
calculates a membrane potential;
calculates an electric field;
updates, by the processor, the kinetic transition rate and state by the calculated ion concentration, membrane potential, and electric field; and
outputs, by the processor, an intracellular voltage and/or ion concentration, for at least one cell.

16. The method of claim 15, wherein each cell within the array of cells further comprises:
an intracellular chemical substance concentration, a membrane chemical substance permeability, and an extracellular chemical substance concentration;
wherein the assigning further comprises:
assigning a kinetic state and transition rate to the chemical substance permeability; and
wherein the iteration further comprises:
calculating a change in chemical substance concentration over time, t;
updating, by the processor, the kinetic transition rate and state by the calculated chemical substance concentration; and
outputting, by the processor, a chemical substance concentration for at least one cell.

17. The method of claim 15, wherein the membrane ion permeability or chemical substance permeability is calculated using a GHK or Nernst Planck flux equation.

18. The method of claim 15, wherein at least one of the intracellular ion concentrations, extracellular ion concentration, ionic permeabilities, intracellular chemical substance concentrations, extracellular chemical substance concentration, or chemical substance permeabilities, is received by the processor.

19. The method of claim 15, wherein the cell comprises a perimeter having multiple facets, wherein a facet of the perimeter is opposed by one or more geometrically equivalent facet(s) of an adjacent cell and/or an adjacent boundary of the array, wherein the boundary of the array comprises a large extracellular space, wherein optionally the perimeters of a 2D array of cells each comprises a hexagonal planar geometry, and wherein a facet of the perimeter comprises a small extracellular space shared with an adjacent facet.

20. The method of claim 15, wherein the cell further comprises a plurality of sub compartments, wherein the processor assigns an intracellular ion concentration to the sub compartment, and wherein the iteration further comprises calculating a Nernst Planck flux equation between any two sub compartments.

21. The method of claim 15, wherein outputting comprises at least one of displaying a number for an intracellular voltage for at least one cell, displaying a graph comprising an intracellular voltage for at least one cell, or displaying an intracellular voltage over time, T.

22. The method of claim 15, wherein the array of cells is 2D or 3D.

23. The method of claim 15, wherein the array of cells, ionic permeability, chemical substance permeability, intracellular ion concentration, extracellular ion concentration, extracellular chemical substance concentration, intracellular chemical substance concentration or combinations thereof corresponds to an injured or an uninjured tissue, and wherein the array of cells, ionic permeability, intracellular ion concentration, extracellular ion concentration, extracellular chemical substance concentration, or intracellular chemical substance concentration or combinations thereof of cells of the tissue is determined by imaging, electrophysiological recordings, measurements of gene expression, or combinations thereof.

24. The method of claim 23, wherein the tissue is selected from the group consisting essentially of an internal organ, a digit, a limb, a muscle, skin, nose, eyes, ears, and brain.

25. The method of claim 15, wherein the ionic permeability of at least one cell is modified by the pharmacological properties of one or more drugs, an electric field, or a gene therapy.

26. An in silico network of virtual cells comprising a digital tissue model represented as a differentiable reaction-diffusion kinetic network, said digital tissue model having at least one bistable virtual cell bioelectrically coupled to a plurality of interconnected virtual cells and virtual environmental compartments, wherein the at least one bistable virtual cell exhibits a membrane ionic permeability that is associated with an artificial ion channel, said digital tissue model being further characterized as capable of propagating a voltage change across the plurality of interconnected virtual cells when a state of the at least one bistable virtual cell is switched from a depolarized state to a hyperpolarized state or vice versa.

27. A method of identifying molecular bioelectric targets for potential bioelectric manipulation or electroceutical intervention comprising interrogating an in silico network of virtual cells, which network comprises a digital twin of a living tissue, by conducting bioelectric simulations in combination with a search algorithm, which simulations iteratively fit and optimize a given bioelectric state to match a predefined bioelectric state under conditions that reveal molecular bioelectric targets that are capable of influencing, restoring, or redirecting bioelectric signaling, thereby enabling cellular decision-making.
